(19) United States
(12) Patent Application Publication
(10) Pub. No.: US 2011/0105324 A1
(43) Pub. Date: May 5, 2011

(54) Herbicidal Compounds Based on N-Azinyl-N-Phenylsulfonylureas

(75) Inventors: Christoph Hugh Rosinger, Hofheim (DE); Dieter Feucht, Eschborn (DE); Klaus-Helmut Mueller, Duesseldorf (DE); Isolde Haeuser-Hahn, Leverkusen (DE); Jan Dittgen, Frankfurt am Main (DE); Ernst Rudolf Gesing, Erkrath (DE); Christian Waldraff, Bad Vibel (DE)

(73) Assignee: BAYER CROPSCIENCE AG, MONHEIM (DE)

(21) Appl. No.: 12/937,319

(22) PCT Filed: Apr. 11, 2009
(86) PCT No.: PCT/EP09/02698

§ 371 Date: Jan. 4, 2011

(30) Foreign Application Priority Data
Apr. 19, 2008 (EP) 0807674.8

(51) Int. Cl. A01N 25/32; C07D 239/42; C07C 311/29; A01N 41/06; C07D 251/16; C07D 251/22; A01P 13/00

(52) U.S. Cl. 504/103; 544/332; 564/90; 504/148; 544/219; 544/216

(57) ABSTRACT

What is described are compounds of the formula (I)

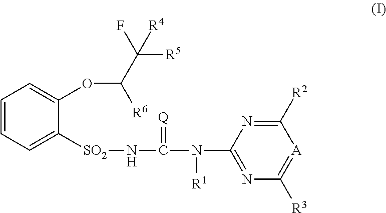

in which the respective substituents have the meanings given in the description. The compounds of the formula (I) can be used as herbicides and plant growth regulators.

The present invention relates to N-azinyl-N′-phenylsulfonylureas. The present invention furthermore relates to mixtures of the abovementioned urea derivatives with other herbicides and/or safeners. Moreover, the present invention relates to processes for the preparation of the abovementioned urea derivatives and to the use of these compounds as herbicides and plant growth regulators alone and in admixture with safeners and/or in admixture with other herbicides, in particular to their use for controlling plants in specific plant crops or as plant protection regulators.

Herbicidally active N-azinyl-N′-arylsulfonylureas having alkoxy groups as substituents in the aryl moiety, where the alkoxy groups may optionally be substituted once again, are known. Substituents mentioned for the alkoxy radicals in question are, for example halogens (cf. DE 41 28 441 A, EP 0 098 569 A, EP 0 023 422 A, EP 0 082 108 A, EP 0 122 231 A, U.S. Pat. No. 4,546,179, EP 0 147 365 A, EP 0 132 230 A, EP 0 124 295 A and U.S. Pat. No. 4,563,211).

EP 0 304 282 A, EP 0 101 407 A and EP 0 107 624 A disclose processes for preparing corresponding N-azinyl-N′-arylsulfonylureas having optionally substituted, in particular halogen-substituted, alkoxy groups.

Furthermore, it is known that certain N-azinyl-N′-arylsulfonylureas having haloalkoxy groups and an additional iodine substituent in the aryl moiety, such as, for example, N-[(4,6-dimethoxypyrimidin-2-yl)carbamoyl]-2-iodo-6-(2,2,2-trifluoro-1-methylethoxy)benzenesulfonamide, N-[(4-chloro-6-methoxypyrimidin-2-yl)carbamoyl]-2-iodo-6-(2,2,2-trifluoro-1-methylethoxy)benzenesulfonamide, N-[(4,6-dimethylpyrimidin-2-yl)carbamoyl]-2-iodo-6-(2,2,2-trifluoro-1-methylethoxy)benzene-sulfonamide, N-[(4,6-dimethoxy-1,3,5-triazin-2-yl)carbamoyl]-2-iodo-6-(2,2,2-trifluoro-1-methylethoxy)benzenesulfonamide and 2-iodo-N-[(4-methoxy-6-methyl-1,3,5-triazin-2-yl)carbamoyl]-6-(2,2,2-trifluoro-1-methylethoxy)benzenesulfonamide, have herbicidal properties (cf. WO 2006/114220).

Also known are certain herbicidally active N-azinyl-N′-arylsulfonylureas which are substituted in the aryl moiety by haloalkoxy groups without additional further substituents (cf. EP 0 023 422 A, EP 0 158 600 A and EP 0 237 480 A).

The active compounds which are already known from the abovementioned specifications have disadvantages when used, be it
(a) that they have no or else only an insufficient herbicidal activity against harmful plants,
(b) that only an unduly narrow spectrum of harmful plants can be controlled, or
(c) that they have an unduly low selectivity in crops of useful plants.

It is therefore desirable to provide alternative chemical active compounds based on corresponding sulfonylurea derivatives which can be employed as herbicides or plant growth regulators and with which certain advantages in comparison with the priorart systems are associated.

Accordingly, it is the general object of the present invention to provide corresponding alternative sulfonylurea derivatives which can be used as herbicides or plant growth regulators, in particular with satisfactory activity against harmful plants, covering a broad spectrum of harmful plants and/or with high selectivity in crops of useful plants. These sulfonylurea derivatives should preferably display an improved property profile, in particular better herbicidal activity against harmful plants, a broader spectrum of harmful plants covered and/or higher selectivity in crops of useful plants, than the sulfonylurea derivatives known from the prior art.

The present invention now provides novel N-azinyl-N′-phenylsulfonylureas of the formula (I)

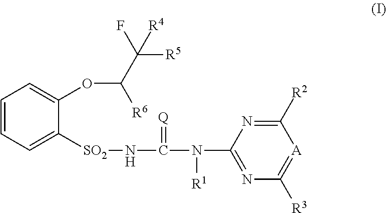

in which
A is selected from the group consisting of nitrogen and CR⁷;
   where
   R⁷ is selected from the group consisting of hydrogen, alkyl, halogen and haloalkyl;
R¹ is selected from the group consisting of hydrogen and an optionally substituted radical from the group consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted cycloalkyl, optionally halogen-substituted alkoxy, optionally halogen-substituted alkylthio, optionally halogen-substituted alkylamino and optionally halogen-substituted dialkylamino;
R³ is selected from the group consisting of hydrogen, halogen, optionally halogen-substituted alkyl, optionally halogen-substituted cycloalkyl, optionally halogen-substituted alkoxy, optionally halogen-substituted alkylthio, optionally halogen-substituted alkylamino and optionally halogen-substituted dialkylamino;
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl;
Q is selected from the group consisting of oxygen and sulfur, in particular oxygen,

and salts of compounds of the formula (I).

A first embodiment of the present invention comprises compounds of the formula (I) in which

A is preferably selected from the group consisting of nitrogen and CH.

A second embodiment of the present invention comprises compounds of the formula (I) in which
R¹ is preferably selected from the group consisting of hydrogen, alkyl, alkoxy, alkoxyalkyl, alkenyl and alkynyl, where the radicals may be unsubstituted or carry one or more halogen atoms,
R¹ is particularly preferably selected from the group consisting of hydrogen, methyl, ethyl, methoxy, methoxymethyl and ethoxy,
R¹ is very particularly preferably selected from the group consisting of hydrogen and methyl,

and
R¹ is especially preferably hydrogen.

A third embodiment of the present invention comprises compounds of the formula (I) in which
R² is preferably selected from the group consisting of hydrogen, halogen, alkyl, cycloalkyl, alkoxy, alkylthio, alkylamino and dialkylamino, where the radicals may be unsubstituted or carry one or more halogen atoms,
R² is particularly preferably selected from the group consisting of hydrogen, chlorine, methyl, ethyl, trifluoromethyl, cyclopropyl, methoxy, ethoxy, trifluoroethoxy, difluoromethoxy, methylthio, methylamino and dimethylamino,

and
R² is especially preferably selected from the group consisting of hydrogen, chlorine, methyl, methoxy, methylthio and dimethylamino.

A fourth embodiment of the present invention comprises compounds of the formula (I) in which
R³ is preferably selected from the group consisting of hydrogen, halogen, alkyl, cycloalkyl, alkoxy, alkylthio, alkylamino and dialkylamino, where the radicals may be unsubstituted or carry one or more halogen atoms,
R³ is particularly preferably selected from the group consisting of hydrogen, chlorine, methyl, ethyl, trifluoromethyl, cyclopropyl, methoxy, ethoxy, trifluoroethoxy, difluoromethoxy, methylthio, methylamino and dimethylamino

and
R³ is especially preferably selected from the group consisting of hydrogen, methyl, methoxy, trifluoroethoxy and dimethylamino.

A fifth embodiment of the present invention comprises compounds of the formula (I) in which
R⁴ is preferably selected from the group consisting of hydrogen, fluorine, chlorine, bromine, methyl, difluoromethyl and trifluoromethyl;
R⁴ is particularly preferably selected from the group consisting of hydrogen, fluorine and trifluoromethyl;

and
R⁴ is especially preferably fluorine.

A sixth embodiment of the present invention comprises compounds of the formula (I) in which
R⁵ is preferably selected from the group consisting of hydrogen, fluorine, chlorine, bromine, methyl, difluoromethyl and trifluoromethyl;
R⁵ is particularly preferably selected from the group consisting of hydrogen, fluorine and trifluoromethyl;

and
R⁵ is especially preferably fluorine.

A seventh embodiment of the present invention comprises compounds of the formula (I) in which
R⁶ is preferably selected from the group consisting of optionally halogen-substituted C₁-C₃-alkyl and C₂-C₄-alkenyl
R⁶ is particularly preferably selected from the group consisting of optionally fluorine- or chlorine-substituted C₁-C₂-alkyl

and

R⁶ is especially preferably selected from the group consisting of optionally fluorine-substituted methyl and ethyl.

The compounds of the formula (I) according to the invention have a substituent of the structure

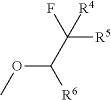

at the aromatic ring. This substituent as component of the compounds of the formula (I) has not been disclosed in the prior art.

Within the scope of these embodiments of the present invention, it is possible to combine the individual general, preferred and especially preferred meanings for the substituents R¹ to R⁶, Q and A as desired. This means that the present invention comprises compounds of the formula (I) in which for example the substituent R¹ has a preferred meaning and the substituents R² to R⁶ have the general meanings, or else for example that the substituent R² has a preferred meaning, the substituent R³ an especially preferred meaning, and the remaining substituents have the general meanings. These individual combinations are not mentioned expressly for reasons of clarity, but must be considered as being comprised by the present invention.

In a further aspect of the present invention, the compound of the formula (I) has the following structure (la) in which R⁴ and R⁵ are fluorine and R⁶ is methyl:

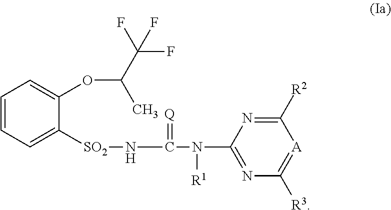

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ib) in which R⁴ and R⁵ are fluorine and R⁶ is ethyl:

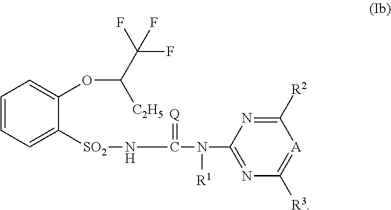

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ic) in which R⁴, R⁵ are fluorine and R⁶ is trifluoromethyl:

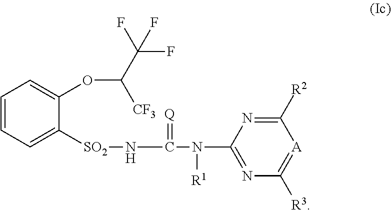

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Id) in which R⁴ and R⁵ are fluorine and R⁶ is n-C₃H₇:

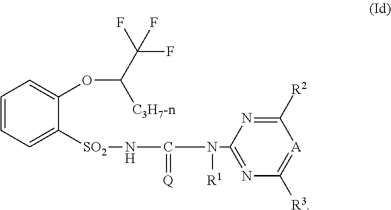

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ie) in which R⁴ and R⁵ are fluorine and R⁶ is allyl:

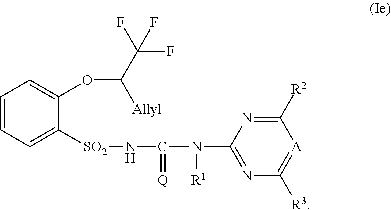

The remaining substituents R¹, R², R³, A and Q have the meanings defined above as general, preferred, particularly preferred and especially preferred for the compounds of the formula (I).

In a further aspect of the present invention, the compound of the formula (I) has the following structure (If) in which R⁴ and R⁵ are hydrogen and R⁶ is CH₂F:

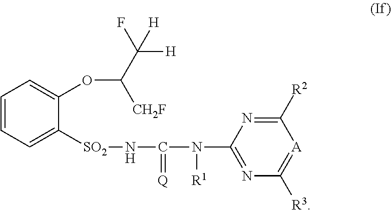

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ig) in which R⁴ and R⁵ are hydrogen and R⁶ is CH₂Cl:

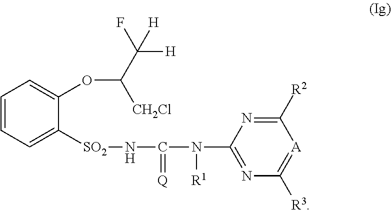

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ih) in which R⁴ is fluorine, R⁵ is CHF₂ and R⁶ is CH₃:

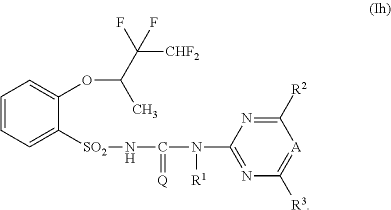

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ii) in which R⁴ is fluorine, R⁵ is CF₃ and R⁶ is C₂H₅:

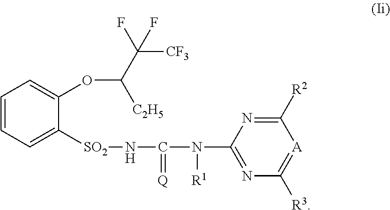

The remaining substituents R¹, R², R³, A and Q have the meanings defined above as general, preferred, particularly preferred and especially preferred for the compounds of the formula (I).

In a further aspect of the present invention, the compound of the formula (I) has the following structure (ID in which R⁴ and R⁵, hydrogen and R⁶ is CH₃:

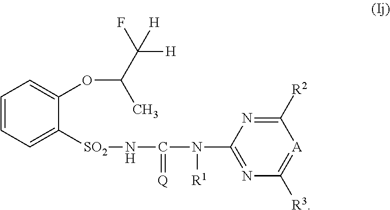

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Ik) in which R⁴ is hydrogen, R⁵ is fluorine and R⁶ is CH₃:

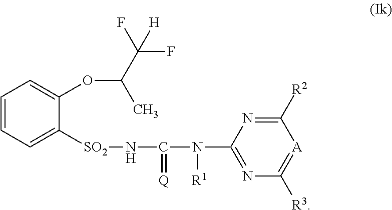

The remaining substituents R¹, R², R³, A and Q have the meanings defined above as general, preferred, particularly preferred and especially preferred for the compounds of the formula (I).

In a further aspect of the present invention, the compound of the formula (I) has the following structure (Im) in which R⁴ is fluorine, R⁵ is CF₃ and R⁶ is CH₃:

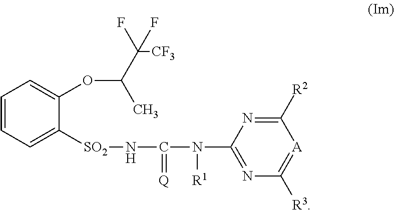

The remaining substituents R¹, R², R³, A and Q have the meanings defined above as general, preferred, particularly preferred and especially preferred for the compounds of the formula (I).

In the compounds of the formula (I), the substituents and radicals R¹ to R⁶, Q and A have the general, preferred, particularly preferred, especially preferred and very especially preferred meanings above.

The present invention preferably also relates to the lithium, sodium, potassium, magnesium, calcium, ammonium, C₁-C₄alkylammonium, di(C₁-C₄-alkyl)ammonium, tri(C₁-C₄-alkyl)ammonium, tetra(C₁-C₄-alkyl)ammonium, tri(C₁-C₄-alkyl)sulfonium, C₅- or C₆-cycloalkylammonium, di(C₁-C₂-alkyl)benzylammonium and tri(C₁-C₂-alkyl)benzylammonium salts of compounds of the formula (I) in which R¹ to R⁶, A and Q have the above general, preferred, especially preferred and particularly preferred meanings and which can be prepared by generally customary methods.

In addition, the compounds of the formula (I) can where appropriate form salts by addition reaction of a suitable inorganic or organic acid, such as, for example, HCl, HBr, H₂SO₄ or HNO₃, but also oxalic acid or sulfonic acids, onto a basic group such as, for example, amino or alkylamino. Suitable substituents which are present in deprotonated form, such as, for example, sulfonic acids or carboxylic acids, can form internal salts with groups which can be protonated in turn, such as amino groups. Salts can also be formed by replacing, in the case of suitable substituents such as, for example, sulfonic acids or carboxylic acids, the hydrogen by a cation which is suitable for the agrochemical sector. Examples of these salts are metal salts, in particular alkali metal salts or alkaline earth metal salts, in particular sodium and potassium salts, or else ammonium salts, salts with organic amines or quaternary ammonium salts with cations of the formula [NRR′R″R′″]⁺, in which R to R′″ in each case independently of one another represent an organic radical, in particular alkyl, aryl, aralkyl or alkylaryl.

In the formula (I) and all other formulae in the present invention, the radicals alkyl, alkoxy, haloalkyl, alkoxyalkyl, alkenyl, alkynyl, haloalkoxy, alkylamino, dialkylamino, alkylthio and haloalkylthio and the corresponding unsaturated and/or substituted radicals in the carbon skeleton can in each case be straight-chain or branched. Unless otherwise specified, the lower carbon skeletons, for example those with 1 to 6 carbon atoms, in particular 1 to 4 carbon atoms, or, in the case of unsaturated groups, having 2 to 6 carbon atoms, in particular 2 to 4 carbon atoms, are preferred among these radicals. Alkyl radicals, also in the composite meanings such as alkoxy, haloalkyl and the like, are, for example, methyl, ethyl, propyls such as n- or i-propyl, butyls such as n-, iso- or tert-butyl, pentyls such as n-pentyl, isopentyl or neopentyl, hexyls such as n-hexyl, i-hexyl, 3-methylpentyl, 2,2-dimethylbutyl or 2,3-dimethylbutyl, heptyls such as n-heptyl, 1-methylhexyl or 1,4-dimethylpentyl; alkenyl and alkynyl radicals have the meaning of the unsaturated radicals which are possible and which correspond to the alkyl radicals and which comprise at least one double bond or triple bond, preferably one double bond or triple bond. Alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, ethynyl, propargyl, but-2-yn-1-yl, but-3-yn-1-yl and 1-methylbut-3-yn-1-yl.

Examples of cycloalkyl groups are cyclopropyl, cyclobutyl, cyclopentyl, cyclohexyl, cycloheptyl and cyclooctyl. The cycloalkyl groups may be present in bicyclic or tricyclic form. Cycloalkylalkyl groups have the meanings which are obtained when cycloalkyl groups are combined with alkyl groups.

If haloalkyl groups and haloalkyl radicals of haloalkoxy, haloalkylthio, haloalkenyl, haloalkynyl and the like are specified, the lower carbon skeletons, for example those having 1 to 6 carbon atoms or 2 to 6, in particular 1 to 4, carbon atoms or preferably 2 to 4 carbon atoms, and the corresponding unsaturated and/or substituted radicals in the carbon skeleton are in each case straight-chain or branched in these radicals. Examples are difluoromethyl, 2,2,2-trifluoroethyl, trifluoroallyl, 1-chloroprop-1-yl-3-yl.

The term “halo” is used synonymously with “halogen” according to the invention.

Alkylene groups in these radicals are the lower carbon skeletons, for example those having 1 to 10 carbon atoms, in particular 1 to 6 carbon atoms or preferably 2 to 4 carbon atoms (unless defined otherwise) and the corresponding unsaturated and/or substituted radicals in the carbon skeleton which can in each case be straight-chain or branched. Examples are methylene, ethylene, n- and isopropylene and n-, sec-, iso- and tert-butylene.

Hydroxyalkyl groups as optionally substituted alkyl groups in these radicals are the lower carbon skeletons, for example those having 1 to 6 carbon atoms, in particular 1 to 4 carbon atoms, and the corresponding unsaturated and/or substituted radicals in the carbon skeleton which can in each case be straight-chain or branched. Examples are 1,2-dihydroxyethyl and 3-hydroxypropyl.

Halogen is fluorine, chlorine, bromine or iodine. Haloalkyl, haloalkenyl and haloalkynyl are alkyl, alkenyl or alkynyl which are partially or fully substituted by halogen, preferably by fluorine, chlorine or bromine, in particular by fluorine and/or chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same applies analogously to haloalkenyl and other halogen-substituted radicals.

Aryl is a mono-, bi- or polycyclic aromatic system, for example phenyl or naphthyl, preferably phenyl. Aralkyl is alkyl-substituted aryl, where alkyl and aryl each have the given definitions. Unless defined otherwise, the definition “substituted by one or more radicals” refers to one or more identical or different radicals.

The substituents mentioned by way of example (“first substituent level”) can, if they contain hydrocarbon-comprising moieties, optionally be further substituted therein (“second substituent level”), for example by one of the substituents as defined for the first substituent level. Corresponding further substituent levels are possible. Preferably, the term “substituted radical” only comprises one or two substituent levels.

Preferred in the case of radicals with carbon atoms are those with 1 to 6 carbon atoms, preferably 1 to 4 carbon atoms, in particular 1 or 2 carbon atoms. As a rule, preferred are substituents from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, hydroxyl, nitro and cyano.

When an aryl radical is substituted, this may preferably be phenyl which is monosubstituted or polysubstituted, preferably up to trisubstituted, by identical or different radicals selected from the group consisting of halogen, (C₁-C₄)-alkyl, (C₁-C₄)-alkoxy, (C₁-C₄)-haloalkyl, (C₁-C₄)-haloalkoxy, cyano and nitro, for example o-, m- and p-tolyl, dimethylphenyls, 2-, 3- and 4-chlorophenyl, 2-, 3- and 4-trifluoromethyl and 2-, 3- and 4-trichloromethylphenyl, 2,4-, 3,5-, 2,5- and 2,3-dichlorophenyl, o-, m- and p-methoxyphenyl.

If appropriate, the present compounds of the formula (I) may comprise at least one chiral carbon atom. Such chiral carbon atoms may occur in particular in the substituent

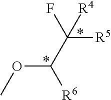

at the carbon atoms marked with an (*).

According to the rules of Cahn, Ingold and Prelog (CIP rules), these carbon atoms may have either the (R) or the (S) configuration.

Embraced by the present invention are compounds of the formula (I) both having (S) and having (R) configuration at the respective chiral carbon atoms; in other words, the present invention encompasses the compounds of the formula (I) in which the carbon atoms in question have in each case independently of one another
(1) an (R) configuration; or
(2) an (S) configuration

If there are two more centers of chirality in the compounds of the formula (I), any desired combinations of the configurations of the chiral centers are possible, i.e. such that
(1) one chiral center has the (R) configuration and the other chiral center has the (S) configuration;
(2) one chiral center has the (R) configuration and the other chiral center has the (R) configuration; and
(3) one chiral center has the (S) configuration and the other chiral center has the (S) configuration.

In particular, the carbon atom marked with an (*) below is chiral, and the present invention embraces both chiral compounds, that is compounds in which the center of chirality in question has the (R) or the (S) configuration:

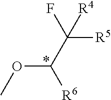

Also embraced by the scope of the present invention are any mixtures of compounds of the formula (I) with carbon atoms having the (R) configuration and carbon atoms having the (S) configuration.

The present invention comprises in particular N-azinyl-N′-phenylsulfonylureas which are present in a stereochemical purity of more than 50% to 100%, in particular more than 60%, especially more than 70%, more especially more than 80%, even more especially more than 90%, even more especially more than 95%, even more especially more than 98%, particularly preferably 100%, in the (R) or (S) configuration with respect to the carbon atom marked with an (*), as shown above.

Depending on the type and attachment of the substituents, the compounds of the formula (I) may contain further centers of chirality in addition to the carbon atoms marked with (*) in formula (I), in which case they are then present as stereoisomers. In the context of the present invention, the definition of the formula (I) fully encompasses all possible stereoisomers, such as enantiomers, diastereomers and Z and E isomers, defined by their specific spatial form, i.e. the present invention comprises both the pure stereoisomers and less pure mixtures thereof.

If, for example, one or more alkenyl groups are present, diastereomers (Z and E isomers) may occur.

If, for example, one or more asymmetric carbon atoms are present, enantiomers and diastereomers may occur.

Corresponding stereoisomers may be obtained from the mixtures resulting from the preparation using customary separation methods, for example by chromatographic separation techniques. It is also possible to prepare stereoisomers selectively by using stereoselective reactions employing optically active starting materials and/or auxiliaries. Accordingly, the invention also relates to all stereoisomers embraced by the formula (I) but not shown in their specific stereoform, and to their mixtures.

For the possible combinations of the various substituents of the formula (I) the general principles of the construction of chemical compounds have to be observed, i.e. the formula (I) does not comprise any compounds known to the person skilled in the art as being chemically impossible.

Preparation of the compounds of the formula (I) according to the invention

The present invention furthermore provides processes for preparing corresponding compounds of the formula (I) and/or their salts.

In a first embodiment of the present invention, the compounds of the formula (I) are prepared by reacting 2-(2-fluoroalkoxy)benzenesulfonamides of the formula (II)

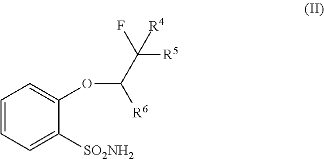

with a heterocyclic (thio)carbamate of the formula (III)

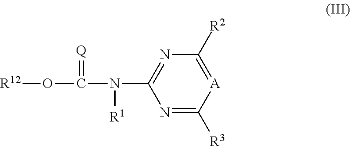

in which R¹² is a substituted or unsubstituted (C₁-C₂₀)-hydrocarbon radical such as aryl or alkyl, preferably optionally substituted phenyl or optionally substituted (C₁-C₄)-alkyl, and in which R¹ to R⁶, Q and A have the above meaning.

The compounds of the formula (II) can be obtained by reacting the compounds of the formula (X) with a strong acid. Suitable strong acids are, for example, mineral acids such as sulfuric acid H₂SO₄ or hydrochloric acid HCl or strong organic acids such as trifluoroacetic acid. The reaction is carried out, for example, at temperatures of from −20° C. to the respective reflux temperature of the reaction mixture, preferably from 0° C. to 40° C. The reaction can be carried out in the absence of a solvent or else in an inert solvent such as, for example, dichloromethane or trichloromethane

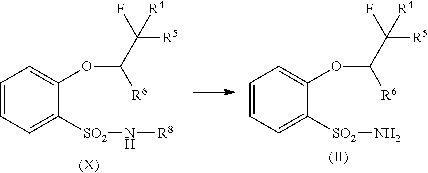

For their part, the compounds of the formula (X) can be obtained by reacting compounds of the formula (XI) with alcohols or their alkali metal salts according to the reaction scheme below, starting with compounds of the formula (XI):

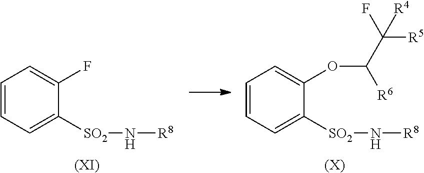

In the formulae (X) and (XI), R⁸ is a branched C₁-C₈-group, preferably a branched C₁-C₄-group, especially preferably a tert-butyl group.

Some of the compounds of the formula (XI) are known and can be prepared by known methods, cf. WO 2006/114221.

Alternatively, the compounds of the formula (II) can also be obtained by exchange of a reactive group, such as, for example, fluorine, from the sulfonamide of the formula (II-a).

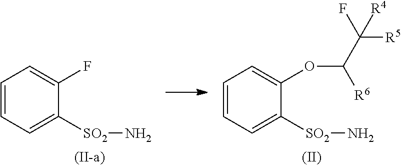

In this reaction, it is also possible to use one or more reaction auxiliaries, such as the customary inorganic or organic bases or acid acceptors. These preferably include alkali metal or alkaline earth metal compounds, for example acetates, amides, carbonates, bicarbonates, hydrides, hydroxides or alkoxides. Particular mention may be made here of potassium carbonate, cesium carbonate, lithium hydroxide, sodium hydroxide and sodium ethoxide, especially sodium hydride. Basic organic nitrogen compounds, for example triethylamine, ethyldiisopropylamine, alkyl-substituted pyridines, 1,4-diazabicyclo[2.2.2]octane (DABCO), 1,5-diazabicyclo[4.3.0]non-5-ene (DBN) or 1,8-diazabicyclo[5.4.0]undec-7-ene (DBU) may also be mentioned. Suitable solvents are, in addition to water, especially inert organic solvents. These include in particular benzene, toluene, xylene, dichloromethane, chloroform, diethyl ether, dioxane, tetrahydrofuran, acetone, acetonitrile, N,N-dimethylformamide, N-methylpyrrolidone or ethyl acetate, and particular mention may be made of diethyl ether, dioxane and tetrahydrofuran. The reaction temperature is generally between −20° C. and the reflux temperature of the solvent used, in particular between 0° C. and the reflux temperature of the solvent used.

In addition to carrying out the reaction in a purely thermal manner, it is also possible to employ microwave energy to accelerate the reaction. To this end, a commercial microwave apparatus designed for chemical purposes may be used. Here, the reactions are generally carried out at temperatures between 20 and 200° C., preferably between 40 and 170° C., using a power of between 20 and 200 watts, preferably between 50 and 180 watts, for a reaction time of between 2 minutes and 60 minutes, preferably between 5 minutes and 45 minutes.

Alternatively, the compounds of the formula (II) can also be obtained by reacting 2-hydroxybenzenesulfonamides of the formulae (II-b) and (X-a) with alcohols of the formula (XII) under Mitsunobu conditions, cf. Journal of Organic Chemistry (2003), 68(21), pp. 8261-8263 and Journal of Combinatorial Chemistry (2002), 4(5), pp. 442-456.

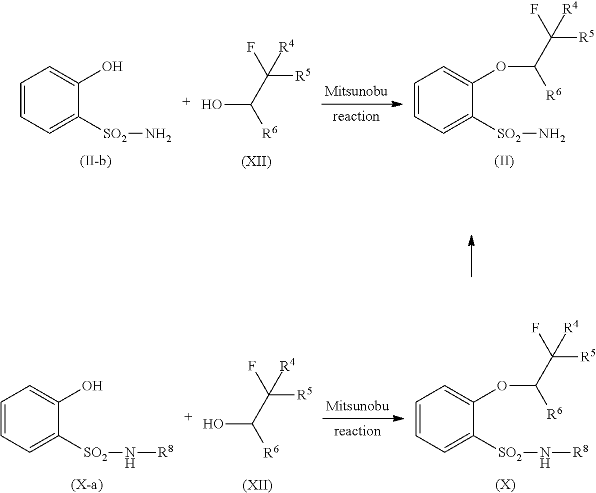

Compound (II-b) is known and commercially available (Chemstep, F-33560 Carbon Blanc, France), compounds of the formula (X-a) can be prepared by known methods, cf. WO 2000/035442 and EP 574090.

In a second embodiment of the present invention, the compounds of the formula (I) are prepared by reacting 2-(2-fluoroalkoxy)benzenesulfonyl iso(thio)cyanates of the formula (IV)

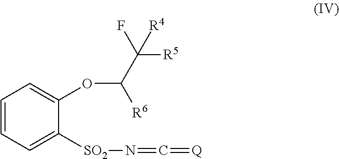

with an aminoheterocycle of the formula (V)

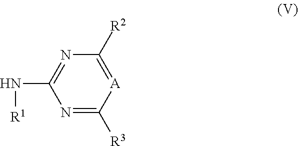

in which R¹ to R⁶, Q and A have the above meaning.

The arylsulfonyl iso(thio)cyanates of the formula (IV) can be prepared by processes known per se from corresponding sulfonamides. Corresponding reactions are known from DE 32 08 189 A, EP 0 023 422 A, EP 0 064 322 A, EP 0 044 807 A and EP 0 216 504 A. The arylsulfonyl iso(thio)cyanates of the formula (IV) are obtained when corresponding arylsulfonamides are reacted with phosgene or thiophosgene, if appropriate in the presence of a reaction auxiliary such as diazabicyclo[2.2.2]octane, and in the presence of a diluent such as toluene, xylene or chlorobenzene at temperatures between 80 and 150° C., and the volatile components are distilled off under reduced pressure after the reaction has ended.

The reaction of the arylsulfonyl iso(thio)cyanates of the formula (IV) with the aminoheterocycles of the formula (V) is carried out, for example, by known processes (cf. WO 2003/91228 A (Scheme 10)).

In a third embodiment of the present invention, the compounds of the formula (I) are prepared by reacting 2-(2-fluoroalkoxy)benzenesulfonyl (thio)carbamates of the formula (VI)

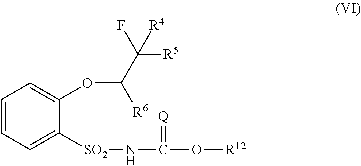

in which R¹² is a substituted or unsubstituted (C₁-C₂₀)-hydrocarbon radical such as aryl or alkyl, preferably optionally substituted phenyl or optionally substituted (C₁-C₄)-alkyl, with an aminoheterocycle of the formula (V)

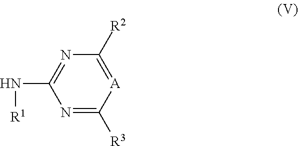

in which R¹ to R⁶, Q and A have the above meaning.

In a fourth embodiment of the present invention, the compounds of the formula (I) are prepared by reacting 2-(2-fluoroalkoxy)benzenesulfonamides of the formula (II)

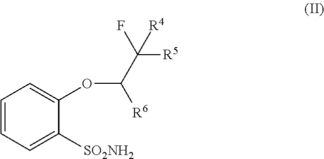

with an iso(thio)cyanate of the formula (VII)

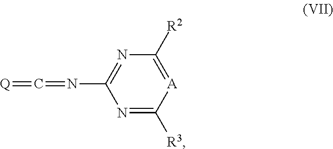

if appropriate in the presence of a reaction auxiliary, where R¹ is hydrogen and R² to R⁶, Q and A have the above meaning.

The iso(thio)cyanates of the formula (VII) are obtained, for example, from aminoheterocycles of the general type (V) where R¹ is hydrogen by treatment with oxalyl chloride or (thio)phosgene (analogously to Angew. Chem. 1971, 83, p. 407; EP 0 388 873 A) The reaction of the iso(thio)cyanates of type (VII) with the sulfonamides of the formula (II) is carried out, for example, analogously to the second embodiment.

In a fifth embodiment of the present invention, the compounds of the formula (I) are prepared by reacting an aminoheterocycle of the formula (V)

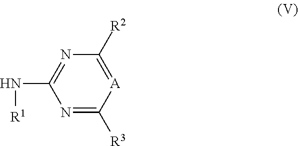

initially under base catalysis with a carbonic ester, for example diphenyl carbonate, and reacting the intermediate of the formula (III) formed

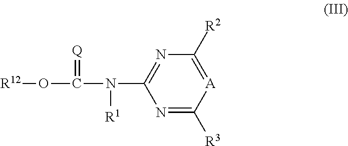

in a one-pot reaction with a 2-(2-fluoroalkoxy)benzenesulfonamide of the formula (II)

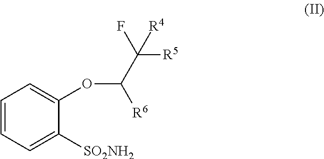

(cf. JP1989221366) in which R¹ to R⁶, R¹², Q and A have the above meaning.

In a sixth embodiment of the present invention, the compounds of the formula (I) are prepared by reacting 2-(2-fluoroalkoxy)benzenesulfonyl halides of the formula (VIII)

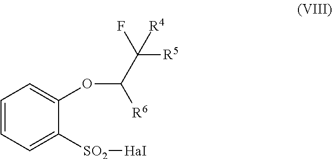

where Hal is a halogen atom, preferably chlorine, with a (thio)cyanate, for example a metal (thio)cyanate, in particular an alkali metal (thio)cyanate such as sodium (thio)cyanate, to give a sulfonyl iso(thio)cyanate of the formula (IV)

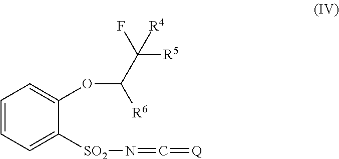

or a solvated (stabilized) derivative thereof, and subsequently with an aminoheterocycle of the formula (V)

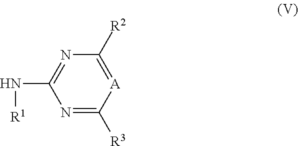

(cf. WO 2003/091228 A and U.S. Pat. No. 5,550,238), where R¹ to R⁶, Q and A are as defined above.

The corresponding sulfonyl chlorides of the formula (VIII-a) can be prepared by known methods from the sulfonamides of the formula (II) (cf. Bull. Kor. Chem. Soc. 1994, 15, 323):

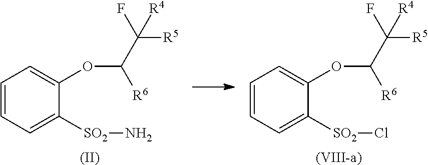

In a seventh embodiment of the present invention, the compounds of the formula (I) where Q=oxygen and R¹=hydrogen are prepared by reacting 2-(2-fluoroalkoxy)benzenesulfonamides of the formula (II)

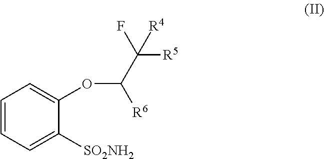

with a heterocyclic biscarbamate of the formula (IX),

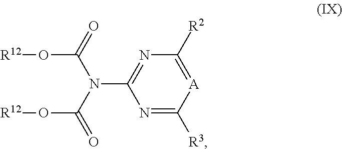

in which R¹² is a substituted or unsubstituted (C₁-C₂₀)-hydrocarbon radical such as aryl or alkyl, preferably optionally substituted phenyl or optionally substituted (C₁-C₄)-alkyl, in the presence of a basic reaction auxiliary, where R² to R⁶ and A have the above meaning (cf. WO 1996/22284 A).

In an eighth embodiment of the present invention, the compounds of the formula (I) are prepared by reacting 2-(2-fluoroalkoxy)benzenesulfonamides of the formula (II)

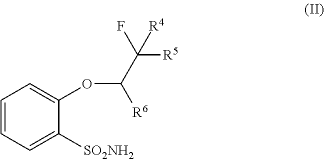

initially under base catalysis with a carbonic ester, for example diphenyl carbonate, and reacting the intermediate of the formula (VI) formed

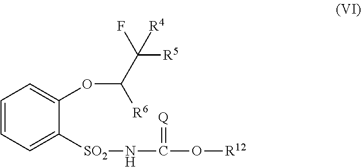

in a one-pot reaction with an aminoheterocycle of the formula (V)

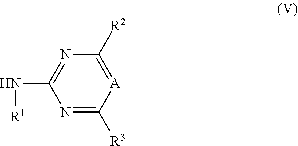

in which R¹ to R⁶, Q and A have the above meaning.

All these processes lead to the compounds of the formula (I) according to the invention.

In the respective process variants mentioned above, use is in each case made of inert solvents. For the purpose of the present invention, inert solvents are solvents which are inert under the reaction conditions in question, i.e. they in particular do not react with the starting materials, but which do not have to be inert under any reaction conditions.

Examples of organic solvents which can be employed in the context of the present invention are aromatic or aliphatic solvents, such as benzene, toluene, xylene, mesitylene, hexane, heptane, octane, cyclohexane, aliphatic and aromatic halogenated hydrocarbons, such as methylene chloride, dichloroethane, chloroform, carbon tetrachloride, chlorobenzene, dichlorobenzene, ethers, such as diethyl ether, dibutyl ether, diisobutyl ether, methyl tert-butyl ether, isopropyl ethyl ether, diisopropyl ether, tetrahydrofuran, and dioxane; furthermore also dimethyl sulfoxide, and acid amide derivatives, such as N,N-dimethylformamide, N,N-dimethylacetamide and N-methyl-2-pyrrolidone, and also carboxylic esters, such as ethyl acetate, or else diglyme, dimethyl glycol; nitriles, such as acetonitrile, propionitrile or butyronitrile, and also ketones, such as acetone, methyl ethyl ketone or cyclohexanone. Particular preference is given to toluene, xylene, dichlorobenzene, chlorobenzene, acetonitrile, acetone, butyronitrile or ethyl acetate. However, the present invention is not limited to the solvents mentioned above in an exemplary manner.

The reaction temperature at which the reactions in accordance with the above embodiments can be carried out may vary within wide limits. Appropriate temperatures are mentioned in the respective embodiments of the reactions. In addition, the reactions can be carried out at a temperature of from 0 to 100° C., preferably from 20 to 70° C.

The reactions of the present invention are generally carried out under atmospheric pressure. However, it is also possible to operate under elevated pressure or under reduced pressure—generally between 0.1 bar and 10 bar.

The processes for preparing the N-azinyl-N′-phenylsulfonylureas of the formula (I) according to the invention are, if appropriate, carried out in the presence of a basic reaction auxiliary.

Suitable basic reaction auxiliaries are all customary inorganic or organic bases. These include, for example, alkali metal or alkaline earth metal hydrides, hydroxides, amides, alkoxides, acetates, carbonates or bicarbonates, such as, for example, lithium hydride, sodium hydride, potassium hydride or calcium hydride, lithium amide, sodium amide or potassium amide, sodium methoxide or potassium methoxide, sodium ethoxide or potassium ethoxide, sodium propoxide or potassium propoxide, aluminum isopropoxide, sodium tert-butoxide or potassium tert-butoxide, sodium hydroxide or potassium hydroxide, ammonium hydroxide, sodium acetate, potassium acetate or calcium acetate, ammonium acetate, sodium carbonate, potassium carbonate or calcium carbonate, ammonium carbonate, sodium bicarbonate or potassium bicarbonate, and also basic organic nitrogen compounds, such as trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl- and 4-methylpyridine, 2,4-dimethyl-, 2,6-dimethyl-, 3,4-dimethyl- and 3,5-dimethylpyridine, 5-ethyl-2-methylpyridine, N-methylpyridine, 4-(N,N-dimethylamino)pyridine, diazabicyclooctane (DABCO), diazabicyclononene (DBN) or diazabicycloundecene (DBU).

Intermediates

The present invention furthermore provides certain intermediates which, according to the synthesis routes shown above, are passed through in the preparation of the compounds of the formula (I) according to the invention.

Accordingly, the present invention also provides, in a first embodiment of the intermediates, compounds of the formula (II)

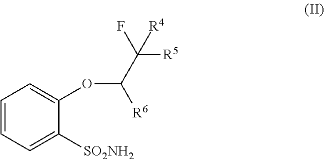

in which the radicals R⁴, R⁵, and R⁶ have the general, preferred and particularly preferred meanings already given above.

The present invention furthermore provides, in a second embodiment of the intermediates, compounds of the formula (IV)

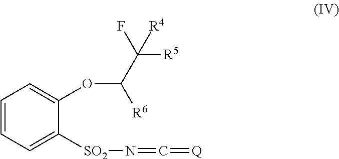

in which the radicals R⁴, R⁵, R⁶ and Q have the general, preferred and particularly preferred meanings already given above.

The present invention furthermore provides, in a third embodiment of the intermediates, compounds of the formula (VIII)

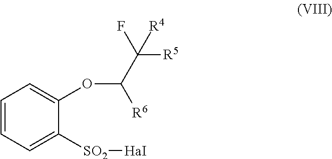

in which the radicals R⁴, R⁵, R⁶ and Hal have the general, preferred and particularly preferred meanings already given above.

The invention furthermore provides, in a fourth embodiment of the intermediates, compounds of the formula (VI)

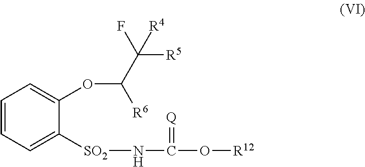

in which the radicals R⁴, R⁵, R⁶, R¹² and Q have the general, preferred and particularly preferred meanings already given above.

The invention furthermore provides, in a fifth embodiment of the intermediates, compounds of the formula (X)

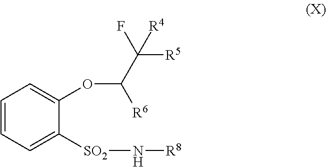

in which the radicals R⁴, R⁵, R⁶ and R⁸ have the general, preferred and particularly preferred meanings already given above.

The present invention also provides compounds of the formulae (II), (IV), (VI), (VIII) and (X) which are present in a stereochemical purity of more than 50% to 100%, in particular more than 60%, especially more than 70%, more especially more than 80%, even more especially more than 90%, even more especially more than 95%, even more especially more than 98%, particularly preferably 100%, in the (R) or (S) configuration with respect to the carbon atom marked with an (*).

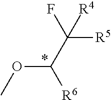

Libraries of compounds of the formula (I) and/or salts thereof which can be synthesized by the aforementioned reactions can also be prepared in a parallel manner, it being possible for this to take place in a manual, partly automated or completely automated manner. In this connection, it is, for example, possible to automate the reaction procedure, the work-up or the purification of the products and/or intermediates. Overall, this is understood as meaning a procedure as described, for example, by D. Tiebes in Combinatorial Chemistry—Synthesis, Analysis, Screening (editor Günther Jung), published by Wiley 1999, on pages 1 to 34.

For the parallel reaction procedure and work-up, it is possible to use a series of commercially available instruments, for example Calypso reaction blocks from Barnstead International, Dubuque, Iowa 52004-0797, USA or reaction stations from Radleys, Shirehill, Saffron Walden, Essex, CB11 3AZ, England or MultiPROBE Automated Workstations from Perkin Elmer, Waltham, Mass. 02451, USA. For the parallel purification of compounds of the formula (I) and salts thereof or of intermediates produced during the preparation, there are available, inter alia, chromatography apparatuses, for example from ISCO, Inc., 4700 Superior Street, Lincoln, Nebr. 68504, USA.

The apparatuses listed lead to a modular procedure in which the individual process steps are automated, but between the process steps manual operations have to be carried out. This can be circumvented by using partly or completely integrated automation systems in which the respective automation modules are operated, for example, by robots. Automation systems of this type can be acquired, for example, from Caliper, Hopkinton, Mass. 01748, USA.

The implementation of single or several synthesis steps can be supported through the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Compounds of the formula (I) and their salts can be prepared not only as in the methods described herein, but also fully or partially by solid-phase-supported methods. For this purpose, individual intermediates or all intermediates of the synthesis or an intermediate adapted to suit the respective procedure are bound to a synthetic resin. Solid-phase-supported synthetic methods are described widely in the specialist literature, for example Barry A. Bunin in “The Combinatorial Index”, Academic Press, 1998 and Combinatorial Chemistry—Synthesis, Analysis, Screening (Editor Günther Jung), published by Wiley, 1999. The use of solid-phase-supported synthetic methods permits a series of protocols known from the literature, which, in turn, can be carried out manually or in an automated fashion. For example, the “teabag method” (Houghten, U.S. Pat. No. 4,631,211; Houghten et al., Proc. Natl. Acad. Sci., 1985, 82, 5131-5135), in which products from IRORI, 11149 North Torrey Pines Road, La Jolla, Calif. 92037, USA, are employed, may be semiautomated. The automation of solid-phase-supported parallel synthesis can be performed successfully, for example, using apparatuses from Argonaut Technologies, Inc., 887 Industrial Road, San Carlos, Calif. 94070, USA or MultiSynTech GmbH, Wullener Feld 4, 58454 Witten, Germany. The reactions can also be carried out, for example, by means of IRORI technology in microreactors from Nexus Biosystems, 12140 Community Road, Poway, Calif. 92064, USA.

Both in the solid phase and in the liquid phase, carrying out individual, or a plurality of, synthesis steps can be supported by using microwave technology. A series of experimental protocols are described in the specialist literature, for example in Microwaves in Organic and Medicinal Chemistry (Editors C. O. Kappe and A. Stadler), published by Wiley, 2005.

The preparation according to the processes described herein produces compounds of the formula (I) and their salts in the form of substance collections which are referred to as libraries. The present invention also provides libraries which comprise at least two compounds of the formula (I) and/or salts thereof.

On account of the herbicidal property of the compounds of the formula (I), the invention also further provides the use of the compounds of the formula (I) according to the invention as herbicides for controlling harmful plants.

The compounds of the formula (I) according to the invention and their salts, hereinbelow together synonymously also referred to as compounds of the formula (I), have an outstanding herbicidal activity against a broad spectrum of economically important monocotyledonous and dicotyledonous harmful plants. The active compounds also have a good effect on perennial harmful plants which produce shoots from rhizomes, root stocks or other perennial organs and which are difficult to control. Here, it is immaterial whether the substances are applied by the pre-sowing, the pre-emergence or the post-emergence method.

The application rate required of the compounds of the formula (I) varies as a function of the external conditions such as, inter alia, temperature, humidity and the nature of the herbicide used. It may vary within wide ranges, for example between 0.001 and 10 000 g/ha or more of active substance; preferably, however, it is between 0.5 and 5000 g/ha, by preference between 0.5 and 1000 g/ha and very especially preferably between 0.5 and 500 g/ha.

Specific mention may be made by way of example of some representatives of the monocotyledonous and dicotyledonous weed flora which can be controlled by the compounds of the formula (I) according to the invention, without the enumeration being restricted to certain species.

On the side of the monocotyledonous weed species, e.g. Agrostis, Alopecurus, Apera, Avena, Brachicaria, Bromus, Dactyloctenium, Digitaria, Echinochloa, Eleocharis, Eleusine, Festuca, Fimbristylis, Ischaemum, Lolium, Monochoria, Panicum, Paspalum, Phalaris, Phleum, Poa, Sagittaria, Scirpus, Setaria, Sphenoclea, and also Cyperus species predominantly from the annual group and on the side of the perennial species Agropyron, Cynodon, Imperata and Sorghum and also perennial Cyperus species are well controlled.

In the case of dicotyledonous weed species, the spectrum of action extends to species such as, for example, Galium, Viola, Veronica, Lamium, Stellaria, Amaranthus, Sinapis, Ipomoea, Matricaria, Abutilon and Sida on the annual side, and Convolvulus, Cirsium, Rumex and Artemisia in the case of the perennial weeds. Moreover, herbicidal effect is observed in the case of dicotyledonous weeds such as Ambrosia, Anthemis, Carduus, Centaurea, Chenopodium, Cirsium, Convolvulus, Datura, Emex, Galeopsis, Galinsoga, Lepidium, Lindernia, Papaver, Portlaca, Polygonum, Ranunculus, Rorippa, Rotala, Seneceio, Sesbania, Solanum, Sonchus, Taraxacum, Trifolium, Urtica and Xanthium.

If the compounds of the formula (I) according to the invention are applied to the soil surface before germination, the weed seedlings are either prevented completely from emerging or else the weeds grow until they have reached the cotyledon stage, but then their growth stops, and, eventually, after three to four weeks have passed, they die completely.

If the active compounds of the formula (I) are applied post-emergence to the green parts of the plants, growth likewise stops drastically a very short time after the treatment, and the weed plants remain at the growth stage at the point of time of application, or they die completely after a certain time, so that in this manner competition by the weeds, which is harmful to the crop plants, is eliminated very early and in a sustained manner.

Although the compounds of the formula (I) according to the invention have excellent herbicidal activity in respect of monocotyledonous and dicotyledonous weeds, crop plants of economically important crops, such as, for example, wheat, barley, rye, rice, corn, sugarbeet, cotton, rapeseed and soybean, are only damaged negligibly, if at all. This is why the present compounds are highly suitable for the selective control of unwanted plant growth in crops of agriculturally useful plants.

In addition, the substances of the formula (I) according to the invention have excellent growth regulatory properties in crop plants. They engage in the plant metabolism in a regulatory fashion and can therefore be employed for the influencing, in a targeted manner, of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops since lodging can be reduced, or prevented completely, hereby.

By virtue of their herbicidal and plant-growth-regulatory properties, the active compounds can also be employed for controlling harmful plants in crops of known genetically modified plants or genetically modified plants still to be developed. In general, the transgenic plants are distinguished by especially advantageous properties, for example by resistances to certain pesticides, mainly certain herbicides, resistances to plant diseases or causative organisms of plant diseases, such as certain insects or microorganisms such as fungi, bacteria or viruses. Other specific characteristics relate, for example, to the harvested material with regard to quantity, quality, storability, composition and specific constituents. Thus, transgenic plants are known whose starch content is increased, or whose starch quality is altered, or those where the harvested material has a different fatty acid composition. Other particular properties may be tolerance or resistance to abiotic stressors, for example heat, low temperatures, drought, salinity and ultraviolet radiation.

Preferred is the use of the compounds of the formula (I) according to the invention or their salts in economically important transgenic crops of useful plants and ornamentals, for example of cereals such as wheat, barley, rye, oats, millet/sorghum, rice, manioc and corn, or else crops of sugar beet, cotton, soybeans, oilseed rape, potato, tomato, pea and other vegetables.

Preferably, the compounds of the formula (I) can be employed as herbicides in crops of useful plants which are resistant, or have been made resistant by recombinant means, to the phytotoxic effects of the herbicides.

Traditional ways for generating novel plants which, in comparison with existing plants, have modified properties consist for example in classical breeding methods and the generation of mutants. Alternatively, it is possible to generate novel plants with modified properties with the aid of recombinant methods (see, for example, EP 0221044, EP 0131624). For example, the following have been described in several cases:
recombinant modifications of crop plants for modifying the starch which is synthesized in the plants (for example WO 92/11376, WO 92/14827, WO 91/19806),
transgenic crop plants which are resistant to certain herbicides of the glufosinate type (cf., for example, EP 0242236, EP 242246) or of the glyphosate type (WO 92/000377) or of the sulfonylurea type (EP 0257993, U.S. Pat. No. 5,013,659),
transgenic crop plants, for example cotton, with the ability to produce Bacillus thuringiensis toxins (Bt toxins), which make the plants resistant to certain pests (EP 0142924, EP 0193259),
transgenic crop plants with a modified fatty acid composition (WO 91/13972),
recombinantly modified crop plants with novel constituents or secondary metabolites, for example novel phytoalexins, which bring about increased disease resistance (EP 0309862, EP 0464461),
recombinantly modified plants with reduced photorespiration which feature higher yields and higher stress tolerance (EP 0305398),
transgenic crop plants which produce pharmaceutically or diagnostically important proteins (“molecular pharming”),
transgenic crop plants which are distinguished by higher yields or better quality,
transgenic crop plants which are distinguished by a combination for example of the abovementioned novel properties (“gene stacking”).

A large number of molecular-biological techniques with the aid of which novel transgenic plants with modified properties can be generated are known in principle, see, for example, I. Potrykus and G. Spangenberg (eds.) Gene Transfer to Plants, Springer Lab Manual (1995), Springer Verlag Berlin, Heidelberg or Christou, “Trends in Plant Science” 1 (1996) 423-431).

To carry out such recombinant manipulations, it is possible to introduce, into plasmids, nucleic acid molecules which permit a mutagenesis or a sequence modification by recombining DNA sequences. With the aid of standard methods, for example, it is possible to carry out base substitutions, to remove part-sequences or to add natural or synthetic sequences. To link the DNA fragments to each other, it is possible to add adapters or linkers to the fragments, see, for example, Sambrook et al., 1989, Molecular Cloning, A Laboratory Manual, 2nd Ed., Cold Spring Harbor Laboratory Press, Cold Spring Harbor, N.Y.; or Winnacker “Gene and Klone” [Genes and Clones], VCH Weinheim 2nd Edition 1996.

The generation of plant cells with a reduced activity of a gene product can be achieved for example by expression of at least one corresponding antisense RNA, a sense RNA for obtaining a cosuppression effect or the expression of at least one suitably constructed ribozyme which specifically cleaves transcripts of the abovementioned gene product.

For this, it is possible firstly to use DNA molecules which comprise the entire coding sequence of a gene product including any flanking sequences which may be present, or else DNA molecules which only comprise parts of the coding sequence, but these parts must be sufficiently long for bringing about an antisense effect in the cells. Another possibility is the use of DNA sequences which have a high degree of homology to the coding sequences of a gene product, but are not entirely identical.

When expressing nucleic acid molecules in plants, the protein synthesized can be localized in any compartment of the plant cell. To achieve localization in a particular compartment, however, it is possible for example to link the coding region to DNA sequences which ensure the localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). Expression of the nucleic acid molecules may also take place in the organelles of the plant cells.

The transgenic plant cells can be regenerated by known techniques to give intact plants. The transgenic plants may, in principle, take the form of plants of any plant species, i.e. both monocotyledonous and dicotyledonous plants.

Thus, it is possible to obtain transgenic plants which feature modified characteristics due to overexpression, suppression or inhibition of homologous (=natural) genes or gene sequences or by expressing heterologous (=foreign) genes or gene sequences.

The compounds of the formula (I) according to the invention can preferably be employed in transgenic crops which are resistant to growth substances, such as, for example, dicamba, or against herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosates, glufosinates or benzoylisoxazoles and analogous active compounds, respectively.

When the active compounds of the formula (I) according to the invention are employed in transgenic crops, they show effects against harmful plants which can also be observed in other crops, but frequently also effects which are specific to the application in the respective transgenic crop, for example a modified or specifically widened weed spectrum which can be controlled, modified application rates which can be employed, preferably good combining ability with the herbicides to which the transgenic crop is resistant, and an effect on growth and yield of the transgenic crop plants.

The invention therefore also relates to the use of the compounds of the formula (I) according to the invention as herbicides for controlling harmful plants in transgenic crop plants.

The compounds of the formula (I) can be formulated in various ways, depending on the prevailing biological and/or chemical-physical parameters. The following are examples of possible formulations: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for broadcasting and soil application, granules (GR) in the form of microgranules, spray granules, absorption granules and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [Chemical Technology], Volume 7, C. Hauser Verlag Munich, 4th Edition 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives are likewise known and are described, for example, in: Watkins, “Handbook of Insecticide Dust Diluents and Carriers”, 2nd Ed., Darland Books, Caldwell N.J., H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schonfeldt, “Grenzflächenaktive Äthylenoxidaddukte” [Interface-active Ethylene Oxide Adducts], Wiss. Verlagsgesell., Stuttgart 1976; Winnacker-Küchler, “Chemische Technologie”, Volume 7, C. Hauser Verlag Munich, 4th Edition 1986.

Based on these formulations, it is also possible to prepare combinations with other pesticidally active compounds such as, for example, insecticides, acaricides, herbicides, fungicides, and also with safeners, fertilizers and/or growth regulators, for example in the form of a ready mix or a tank mix.

Wettable powders are preparations which are uniformly dispersible in water and which, besides a diluent or inert compound, also comprise ionic and/or nonionic surfactants (wetting agents, dispersants) in addition to the active compound, for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium ligninsulfonate, sodium 2,2′-dinaphthylmethane-6,6′-disulfonate, sodium dibutylnaphthalenesulfonate or else sodium oleoylmethyltaurite. To prepare the wettable powders, the herbicidal active compounds are ground finely, for example in customary apparatuses such as hammer mills, blower mills and air-jet mills, and simultaneously or subsequently mixed with the formulation auxiliaries.

Emulsifiable concentrates are prepared by dissolving the active compound in an organic solvent, for example butanol, cyclohexanone, dimethylformamide, xylene or else higher-boiling aromatics or hydrocarbons or mixtures of the organic solvents, with addition of one or more ionic and/or nonionic surfactants (emulsifiers). Examples of emulsifiers which can be used are: calcium salts of alkylarylsulfonic acids, such as calcium dodecylbenzenesulfonate, or nonionic emulsifiers such as fatty acid polyglycol esters, alkylarylpolyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active compound with finely divided solid materials, for example talc, natural clays such as kaolin, bentonite and pyrophyllite, or diatomaceous earth.

Suspension concentrates may be water- or oil-based. They can be prepared for example by wet-grinding using commercially available bead mills and, if appropriate, an addition of surfactants as have already been listed for example above in the case of the other formulation types.

Emulsions, for example oil-in-water emulsions (EW), can be prepared for example by means of stirrers, colloid mills and/or static mixers using aqueous organic solvents and, if appropriate, surfactants as have already been listed for example above in the case of the other formulation types.

Granules can be prepared either by spraying the active compound onto adsorptive granulated inert material or by applying active compound concentrates to the surface of carriers such as sand, kaolinites or granulated inert material by means of binders, for example polyvinyl alcohol, sodium polyacrylate or else mineral oils. Suitable active compounds can also be granulated in the manner which is conventionally used for the preparation of fertilizer granules, if appropriate as a mixture with fertilizers.

In general, water-dispersible granules are prepared by the customary methods such as spray-drying, fluidized-bed granulation, disk granulation, mixing in high-speed mixers and extrusion without solid inert material.

To prepare disk, fluidized-bed, extruder and spray granules, see, for example, the methods in “Spray-Drying Handbook” 3rd Ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, pp. 8-57.

For further details on the formulation of plant protection products, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

In general, the agrochemical preparations comprise from 0.1 to 99% by weight, in particular from 0.1 to 95% by weight, of active compound of the formula (I).

In wettable powders, the active compound concentration amounts to for example approximately 10 to 90% by weight, the remainder to 100% by weight is composed of conventional formulation components. In the case of emulsifiable concentrates, the active compound concentration may be approximately 1 to 90%, preferably from 5 to 80%, by weight. Formulations in the form of dust comprise from 1 to 30% by weight of active compound, preferably in most cases from 5 to 20% by weight of active compound, while sprayable solutions comprise from approximately 0.05 to 80%, preferably from 2 to 50%, by weight of active compound. In the case of water-dispersible granules, the active compound content depends partly on whether the active compound is present in liquid or solid form and on the granulation auxiliaries, fillers and the like which are used. In the case of the water-dispersible granules, the active compound content is, for example, between 1 and 95% by weight, preferably between 10 and 80% by weight.

In addition, the abovementioned active compound formulations comprise, if appropriate, the adhesives, wetters, dispersants, emulsifiers, penetrants, preservatives, antifreeze agents, solvents, fillers, carriers, colorants, antifoam agents, evaporation inhibitors and agents which affect the pH and the viscosity which are customary in each case.

The compounds of the formula (I) or their salts can be employed as such or in the form of their preparations (formulations) as a combination with other pesticidally active compounds such as, for example, insecticides, acaricides, nematicides, herbicides, fungicides, safeners, fertilizers and/or growth regulators, for example as a ready mix or as tank mixes. Combination partners which can be used for the active compounds of the formula (I) according to the invention in mixed formulations or in a tank mix are, for example, known active compounds which are based on the inhibition of, for example,

acetolactate synthase, acetyl-coenzyme A carboxylase, PS I, PS II, HPPDO, phytoene desaturase, protoporphyrinogen oxidase, glutamine synthetase, 5-enolpyruvylshikimate-3-phosphate synthetase or cellulose biosynthesis. Such compounds, and also other usable compounds, with a mechanism of action that is, in some cases, unknown or different, are described, for example, in Weed Research 26, 441-445 (1986), or “The Pesticide Manual”, 11th edition 1997 (hereinafter also abbreviated to “PM”) and 12th edition 2000, The British Crop Protection Council and the Royal Soc. of Chemistry (Publisher), and the literature cited there. Herbicides which are known from the literature and which can be combined with the compounds of the formula (I), include, for example, the following active ingredients (note: the compounds are either referred to by the common name in accordance with the International Organization for Standardization (ISO) or by the chemical name, if appropriate together with a customary code number):

acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; acrolein; alachlor; alloxydim(-sodium); ametryn; amicarbazone, amidochlor, amidosulfuron; aminocyclopyrachlor (CAS-RN: 858956-08-8) aminopyralid, amitrol; AMS, i.e. ammonium sulfamate; anilofos; asulam; atraton; atrazin; azafenidin, azimsulfuron (DPX-A8947); aziprotryn; barban; BAS 516 H, i.e. 5-fluoro-2-phenyl-4H-3,1-benzoxazin-4-one; BCPC; beflubutamid, benazolin(-ethyl); benfluralin; benfuresate; bensulfuron(-methyl); bensulide; bentazone; benzfendizone; benzobicyclon, benzofenap; benzofluor; benzoylprop(-ethyl); benzthiazuron; bifenox; bialaphos; bifenox; bispyribac(-sodium), borax; bromacil; bromobutide; bromofenoxim; bromoxynil; bromuron; buminafos; busoxinone; butachlor; butafenacil, butamifos; butenachlor; buthidazole; butralin; butroxydim, butylate; cacodylic acid; calcium chlorate; cafenstrole (CH-900); carbetamide; carfentrazone(-ethyl); caloxydim, CDAA, i.e. 2-chloro-N,N-di-2-propenylacetamide; CDEC, i.e. 2-chloroallyl diethyldithiocarbamate; chlorflurenol (-methyl); chlomethoxyfen; clethodim; clomeprop; chloramben; chlorazifop-butyl, chlormesulon; chlorbromuron; chlorbufam; chlorfenac; chlorflurecol-methyl; chloridazon; chlorimuron(-ethyl); chloroacetic acid; chlornitrofen; chlorotoluron; chloroxuron; chlorpropham; chlorsulfuron; chlorthal(-dimethyl); chlorthiamid; chlortoluron, cinidon(-methyl and -ethyl), cinmethylin; cinosulfuron; cisanilide; clefoxydim, clethodim; clodinafop and its ester derivatives (e.g. clodinafop-propargyl); clomazone; clomeprop; cloproxydim; clopyralid; clopyrasulfuron(-methyl); cloransulam(-methyl), cresol; cumyluron (JC 940); cyanamide; cyanazine; cycloate; cyclosulfamuron (AC 104); cycloxydim; cycluron; cyhalofop and its ester derivatives (e.g. butyl ester, DEH-112); cyperquat; cyprazine; cyprazole; daimuron; 2,4-D, 2,4-DB, 3,4-DA, 3,4-DB, 2,4-DEB, dalapon; dazomed; desmedipham; desmetryn; di-allate; dicamba; dichlobenil; ortho-dichlorobenzene; para-dichlorobenzene; dichlorprop; dichlorprop-P; diclofop and its esters such as diclofop-methyl; diclosulam, diethatyl(-ethyl); difenoxuron; difenzoquat; difenzoquat-methylsulphate; diflufenican; diflufenzopyr, dimefuron; dimepiperate, dimethachlor; dimethametryn; dimethenamid (SAN-582H); dimethenamid-P; dimethazone, dimexyflam, dimethipin; diemethylarsinic acid; dinitramine; dinoseb; dinoterb; diphenamid; dipropetryn; diquat; diquat-dibromide; dithiopyr; diuron; DNOC; 3,4-DP; DSMA; EBEP; eglinazine-ethyl; EL77, i.e. 5-cyano-1-(1,1-dimethylethyl)-N-methyl-1H-pyrazole-4-carboxamide; endothal; epoprodan, EPTC; esprocarb; ethalfluralin; ethametsulfuron(-methyl); ethidimuron; ethiozin; ethofumesate; ethoxyfen and its esters (e.g. ethyl ester, HN-252); ethoxysulfuron, etobenzanid (HW 52); F5231, i.e. N-[2-chloro-4-fluoro-5-[4-(3-fluoropropyl)-4,5-dihydro-5-oxo-1H-tetrazol-1-yl]phenyl]ethanesulfonamide; fenoprop; fenoxan, fenoxaprop and fenoxaprop-P and their esters, e.g. fenoxaprop-P-ethyl and fenoxaprop-ethyl; fenoxydim; fentrazamide, fenuron; ferrous sulfate; flamprop(-methyl or -isopropyl or -isopropyl-L); flazasulfuron; floazulate, florasulam, fluazifop and fluazifop-P and their esters, e.g. fluazifop-butyl and fluazifop-P-butyl; fluazolate; flucarbazone(-sodium), flucetosulfuron; fluchloralin; flufenacet; flufenpyr(-ethyl); flumetsulam; flumeturon; flumiclorac(-pentyl), flumioxazin (S-482); flumipropyn; fluometuron, fluorochloridone, fluorodifen; fluoroglycofen(-ethyl); flupoxam (KNW-739); flupropacil (UBIC-4243); flupropanate, flupyrsulfuron(-methyl or -sodium), flurenol(-butyl), fluridone; flurochloridone; fluroxypyr(-meptyl); flurprimidol; flurtamone; fluthiacet(-methyl) (KIN-9201); fluthiamide; fomesafen; foramsulfuron; fosamine; furyloxyfen; glufosinate(-ammonium); glyphosate(-isopropylammonium); halosafen; halosulfuron(-methyl) and its esters (e.g. methyl ester, NC-319); haloxyfop and its esters; haloxyfop-P (=R-haloxyfop) and its esters; HC-252; hexazinone; imazamethabenz(-methyl); imazapyr; imazaquin and salts such as the ammonium salt; imazamethapyr, imazamox, imazapic, imazethamethapyr; imazethapyr; imazosulfuron; indanofan, iodomethane; iodosulfuron(methylsodium); ioxynil; isocarbamid; isopropalin; isoproturon; isouron; isoxaben; isoxachlortole, isoxaflutole, isoxapyrifop; karbutilate; lactofen; lenacil; linuron; MAA; MAMA; MCPA; MCPA-2-ethylhexyl; MCPA-thioethyl; MCPB; mecoprop; mecoprop-P; mefenacet; mefluidid; mesosulfuron(-methyl); mesotrione, metamifop; metamitron; metazachlor; methabenzthiazuron; metham; methazole; methoxyphenone; methylarsonic acid; methyldymron; methyl isothiocyanate; metabenzuron, metamifop; methobenzuron; metobromuron; (alpha-)metolachlor; S-metolachlor; metosulam (XRD 511); metoxuron; metribuzin; metsulfuron-methyl; MK-616; MH; molinate; monalide; monocarbamide dihydrogensulfate; monolinuron; monuron; monosulfuron; MSMA; MT 128, i.e. 6-chloro-N-(3-chloro-2-propenyl)-5-methyl-N-phenyl-3-pyridazinamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; nonanoic acid; norflurazon; oleic acid (fatty acid); orbencarb; orthosulfamuron; oryzalin; oxadiargyl (RP-020630); oxadiazon; oxasulfuron, oxaziclomefone, oxyfluorfen; paraquat; paraquat dichloride; pebulate; pelargonic acid, pendimethalin; penoxsulam; pentachlorophenol; pentanochlor; pentoxazone, perfluidone; phenisopham; phenmedipham(ethyl); pethoxamid; picloram; picolinafen, pinoxaden, piperophos; piributicarb; pirifenop-butyl; pretilachlor; primisulfuron(-methyl); potassium arsenite; potassium azide; procarbazone-(sodium), procyazine; prodiamine; profluazol; profluralin; profoxydim; proglinazine(-ethyl); prometon; prometryn; propachlor; propanil; propaquizafop and its esters; propazine; propham; propisochlor; propoxycarbazone(-sodium) (BAY MKH 6561); propyzamide; prosulfalin; prosulfocarb; prosulfuron (CGA-152005); prynachlor; pyraclonil; pyraflufen(-ethyl), pyrasulfotole; pyrazolinate; pyrazon; pyrazosulfuron(-ethyl); pyrazoxyfen; pyribambenz-isopropyl; pyribenzoxim, pyributicarb, pyridafol, pyridate; pyriftalid; pyrimidobac(-methyl), pyrimisulfan, pyrithiobac(-sodium) (KIN-2031); pyroxasulfone; pyroxofop and its esters (e.g. propargyl ester); pyroxsulam (triflosulam); quinclorac; quinmerac; quinoclamine, quinofop and its ester derivatives, quizalofop and quizalofop-P and its ester derivatives, e.g. quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; saflufenacil (CAS-RN: 372137-35-4); secbumeton; sethoxydim; siduron; simazine; simetryn; SN 106279, i.e. 2-[[7-2-chloro-4-(trifluoromethyl)phenoxy]-2-naphthalenyl]oxy]propanoic acid and its methyl ester; SMA; sodium arsenite; sodium azide; sodium chlorate; sulcotrione, sulfentrazon (FMC-97285, F-6285); sulfazuron; sulfometuron(-methyl); sulfosate (ICI-A0224); sulfosulfuron, 2,3,6-TBA; TCA(sodium); tebutam (GCP-5544); tebuthiuron; tefuryltrione, tembotrione, tepraloxydim, terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiafluamide, thiazafluron; thiazopyr (Mon-13200); thidiazimin (SN-24085); thien-carbazone-methyl, thifensulfuron(-methyl); thiobencarb; tiocarbazil; tralkoxydim; tri-allate; triasulfuron; triaziflam, triazofenamide; tribenuron(-methyl); tricamba; tric-lopyr; tridiphane; trietazine; trifloxysulfuron(sodium); trifluralin; triflusulfuron-methyl and esters (e.g. methyl ester, DPX-66037); trihydroxytriazine; trimeturon; tritosulfuron; tropramezone; tsitodef; vernolate; [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester; WL 110547, i.e. 5-phenoxy-1-[3-(trifluoromethyl)phenyl]-1H-tetrazole; UBH-509; D-489; LS 82-556, i.e. [(S)-3-N-(methylbenzyl)carbamoyl-5-propionyl-2,6-lutidine]; KPP-300; NC-324; NC-330; KH-218; DPX-N8189; SC-0774; DOWCO-535; DK-8910; V-53482; PP-600; MBH-001; ET-751, i.e. ethyl [2-chloro-5-(4-chloro-5-difluoromethoxy-1-methyl-1H-pyrazol-3-yl)-4-fluorophenoxy]acetate; KIH-6127, i.e. pyriminobac-methyl; KIH-2023, i.e. bispyribac-sodium; and SYP-249, i.e. ethyl 2-{2-nitro-5-[(2-chloro-4-trifluoromethyl)phenoxy]benzoxy}-3-methyl-3-butenoate; SYN-523.

Of particular interest is the selective control of harmful plants in crops of useful plants and ornamental plants. Although the compounds of the formula (I) according to the invention already have very good to adequate selectivity in many crops, it is in principle possible, in some crops and primarily also in the case of mixtures with other herbicides which are less selective, for phytotoxicities on the crop plants to occur. In this connection, combinations of compounds of the formula (I) according to the invention are of particular interest which comprise the compounds of the formula (I) or their combinations with other herbicides or pesticides and safeners. The safeners which are used in an antidotically effective amount reduce the phytotoxic side-effects of the herbicides/pesticides used, e.g. in economically important crops such as cereals (wheat, barley, rye, corn, rice, millet), sugarbeet, sugarcane, rapeseed, cotton and soybean, preferably cereals. The following groups of compounds are suitable, for example, as safeners for the compounds (I) alone or else in their combinations with further pesticides:
S1) Compounds of the formula (S1),

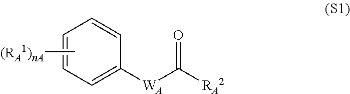

where the symbols and indices have the following meanings:
n_{A} is a natural number from 0 to 5, preferably 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-ring heterocycles having 1 to 3 heteroring atoms from the group consisting of N and O, where at least one N atom and at most one O atom is present in the ring, preferably a radical from the group (W_{A}¹) to (W_{A}⁴),

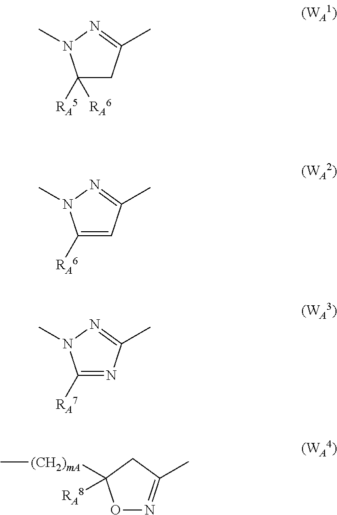

m_{A} is 0 or 1;
R_{A}² is OR_{A}³, SR_{A}³ or NR_{A}³R_{A}⁴ or a saturated or unsaturated 3- to 7-membered heterocycle with at least one N atom and up to 3 heteroatoms, preferably from the group consisting of O and S, which is bonded to the carbonyl group in (S1) via the N atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbon radical, preferably having in total 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹, in which R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different, hydrogen, (C₁-C₈)-alkyl, (C₁-C₈)-haloalkyl, (C₃-C₁₂)-cycloalkyl or substituted or unsubstituted phenyl;

preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds, as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds, as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds, as described, for example, in EP-A-268554;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds, as described in EP-A-174 562 and EP-A-346 620;
e) compounds of the 5-benzyl- or 5-phenyl-2-isoxazoline-3-carboxylic acid type or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds, as described in WO-A-91/08202, or 5,5-diphenyl-2-isoxazoline-carboxylic acid (S1-10) or ethyl 5,5-diphenyl-2-isoxazolinecarboxylate (S1-11) (“isoxadifen-ethyl”) or n-propyl 5,5-diphenyl-2-isoxazolinecarboxylate (S1-12) or of the ethyl 5-(4-fluorophenyl)-5-phenyl-2-isoxazoline-3-carboxylate type (S1-13), as described in the patent application WO-A-95/07897.
S2) Quinoline derivatives of the formula (S2),

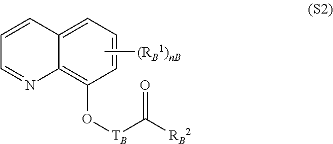

where the symbols and indices have the following meanings:
R_{B}¹ is halogen, (C₁-C₄)-alkyl, (C₁-C₄)-alkoxy, nitro or (C₁-C₄)-haloalkyl;
n_{B} is a natural number from 0 to 5, preferably 0 to 3;
R_{B}² is OR_{B}³, SR_{B}³ or NR_{B}³R_{B}⁴ or a saturated or unsaturated 3- to 7-membered heterocycle having at least one N atom and up to 3 heteroatoms, preferably from the group consisting of O and S, which is joined to the carbonyl group in (S2) via the N atom and is unsubstituted or substituted by radicals from the group consisting of (C₁-C₄)-alkyl, (C₁-C₄)-alkoxy or optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbon radical, preferably having in total 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;

preferably:
a) compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl(5-chloro-8-quinolinoxy)acetate (S2-2), 4-allyloxybutyl (5-chloro-8-quinolinoxy)acetate (S2-3), 1-allyloxyprop-2-yl(5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), its hydrates and salts, for example its lithium, sodium, potassium, calcium, magnesium, aluminum, iron, ammonium, quaternary ammonium, sulfonium or phosphonium salts, as described in WO-A-2002/34048;
b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)-malonate and related compounds, as described in EP-A-0 582 198.
S3) Compounds of the formula (S3)

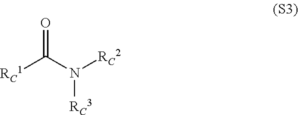

where the symbols and indices have the following meanings:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different, hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;

preferably:
active compounds of the dichloroacetamide type, which are often used as pre-emergence safeners (soil-active safeners), such as, for example, “dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (S3-6),
“AD-67” or “MON 4660” (3-dichloroacetyl-1-oxa-3-azaspiro[4,5]decane) from Nitrokemia or Monsanto (S3-7),
“T1-35” (1-dichloroacetylazepane) from TRI-Chemical RT (S3-8), “diclonon” (dicyclonone) or “BAS145138” or “LAB145138” (S3-9) (3-dichloroacetyl-2,5,5-trimethyl-1,3-diazabicyclo[4.3.0]nonane) from BASF, “furilazole” or “MON 13900” ((RS)-3-dichloroacetyl-5-(2-furyl)-2,2-dimethyl-oxazolidine) (S3-10); and also its (R)-isomer (S3-11).
S4) N-Acylsulfonamides of the formula (S4) and their salts,

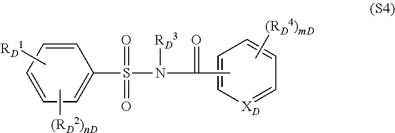

in which the symbols and indices have the following meanings:
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkyl-sulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl (C₅-C₆)-cycloalkenyl, phenyl or 3- to 6-membered heterocyclyl comprising v_{D} heteroatoms from the group consisting of nitrogen, oxygen and sulfur, where the seven last-mentioned radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three last-mentioned radicals are substituted by v_{D} radicals from the group consisting of halogen, hydroxyl, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;

of which preference is given to compounds of the N-acylsulfonamide type, for example of the following formula (S4^{a}), which are known, for example, from WO-A-97/45016,

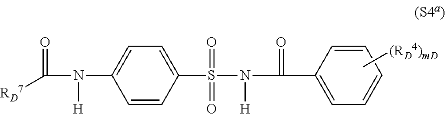

in which
R_{D}⁷ is (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃;
m_{D} is 1 or 2;
v_{D} is 0, 1, 2 or 3;

and

acylsulfamoylbenzamides, e.g. of the following formula (S4^{b}), which are known, for example, from WO-A-99/16744,

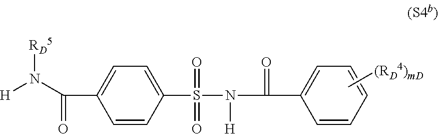

e.g. those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5),

and

compounds of the N-acylsulfamoylphenylurea type of the formula (S4^{c}), which are known, for example, from EP-A-365484,

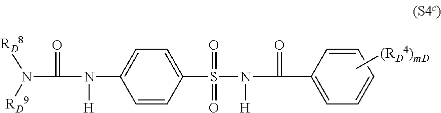

in which
R_{D}⁸ and R_{D}⁹, independently of one another, are hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is 1 or 2;

for example
1-[4-(N-2-methoxybenzoylsulfamoyl)phenyl]-3-methylurea, 1-[4-(N-2-methoxybenzoylsulfamoyl)phenyl]-3,3-dimethylurea, 1-[4-(N-4,5-dimethylbenzoylsulfamoyl)phenyl]-3-methylurea.
S5) Active compounds from the class of hydroxyaromatics and aromatic-aliphatic carboxylic acid derivatives (S5), e.g. ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), e.g. 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydro-quinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

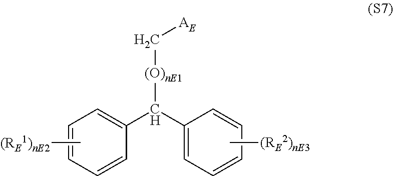

in which the symbols and the indices have the following meanings:
R_{E}¹, R_{E}² independently of one another are halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴
R_{E}³, R_{E}⁴ independently of one another are hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1
n_{E}², n_{E}³ independently of one another are 0, 1 or 2,
preferably:
   diphenylmethoxyacetic acid,
   ethyl diphenylmethoxyacetate,
   methyl diphenylmethoxyacetate (CAS Reg. No. 41858-19-9) (S7-1).
S8) Compounds of the formula (S8), as described in WO-A-98/27049,

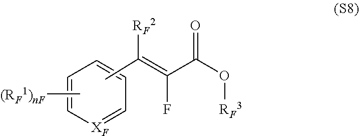

in which
X_{F} is CH or N,
n_{F} if X_{F}=N, is an integer from 0 to 4 and
if X_{F}═CH, is an integer from 0 to 5,
R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl, or aryl, where each of the aforementioned C-containing radicals is unsubstituted or substituted by one or more, preferably up to three, identical or different radicals from the group consisting of halogen and alkoxy, or salts thereof,

preferably compounds in which
X_{F} is CH,
n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl, or aryl, where each of the aforementioned C-containing radicals is unsubstituted or substituted by one or more, preferably up to three, identical or different radicals from the group consisting of halogen and alkoxy, or salts thereof.
S9) Active compounds from the class of the 3-(5-tetrazolylcarbonyl)-2-quinolones (S9), e.g. 1,2-dihydro-4-hydroxy-1-ethyl-3-(5-tetrazolylcarbonyl)-2-quinolone (CAS Reg. No. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formula (S10^{a}) or (S10^{b}), as described in WO-A-2007/023719 and WO-A-2007/023764

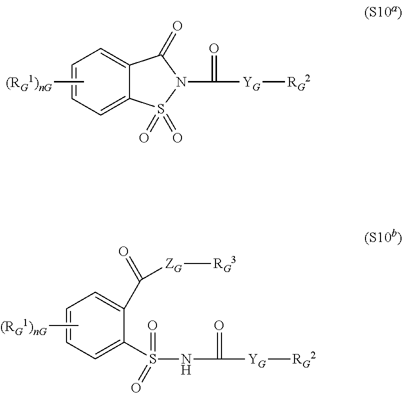

in which
R_{G}¹ is halogen, (C₁-C₄)-alkyl, methoxy, nitro, cyano, CF₃, OCF₃
Y_{G}, Z_{G} independently of one another are O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active compounds of the oxyimino compound type (S11), which are known as seed dressings, such as, for example,
   “oxabetrinil” ((Z)-1,3-dioxolan-2-ylmethoxyimino(phenyl)acetonitrile) (S11-1), which is known as seed dressing safener for millet against metolachlor damage,
   “fluxofenim” (1-(4-chlorophenyI)-2,2,2-trifluoro-1-ethanone O-(1,3-dioxolan-2-ylmethyl)oxime) (S11-2), which is known as seed dressing safener for millet against metolachlor damage, and
   “cyometrinil” or “CGA-43089” ((Z)-cyanomethoxyimino(phenyl)acetonitrile) (S11-3), which is known as seed dressing safener for millet against metolachlor damage.
S12) Active compounds from the class of the isothiochromanones (S12), such as, for example, methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as seed dressing safener for corn against thiocarbamate herbicide damage,
   “fenclorim” (4,6-dichloro-2-phenylpyrimidine) (S13-2), which is known as safener for pretilachlor in sown rice,
   “flurazole” (benzyl 2-chloro-4-trifluoromethyl-1,3-thiazole-5-carboxylate) (S13-3), which is known as seed dressing safener for millet against alachlor and metolachlor damage,
   “CL 304415” (CAS Reg. No. 31541-57-8) (4-carboxy-3,4-dihydro-2H-1-benzopyran-4-acetic acid) (S13-4) from American Cyanamid, which is known as safener for corn against imidazolinone damage,
   “MG 191” (CAS Reg. No. 96420-72-3) (2-dichloromethyl-2-methyl-1,3-dioxolane) (S13-5) from Nitrokemia, which is known as safener for corn,
   “MG-838” (CAS Reg. No. 133993-74-5) (2-propenyl 1-oxa-4-azaspiro[4.5]decane-4-carbodithioate) (S13-6) from Nitrokemia,
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active compounds which, besides a herbicidal effect against harmful plants, also have safener effect on crop plants such as rice, such as, for example,
   “dimepiperate” or “MY-93” (S-1-methyl-1-phenylethyl piperidine-1-carbothioate), which is known as safener for rice against molinate herbicide damage,
   “daimuron” or “SK 23” (1-(1-methyl-1-phenylethyl)-3-p-tolylurea), which is known as safener for rice against imazosulfuron herbicide damage,
   “cumyluron”=“JC-940” (3-(2-chlorophenylmethyl)-1-(1-methyl-1-phenyl-ethyl)urea, see JP-A-60087254), which is known as safener for rice against some herbicide damage,
   “methoxyphenone” or “NK 049” (3,3′-dimethyl-4-methoxybenzophenone), which is known as safener for rice against some herbicide damage,
   “CSB” (1-bromo-4-(chloromethylsulfonyl)benzene) from Kumiai, (CAS Reg. No. 54091-06-4), which is known as safener against some herbicide damage in rice.
S15) Active compounds which are primarily used as herbicides, but also have safener effect on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Some of the safeners are already known as herbicides and thus, besides the herbicidal effect in respect of harmful plants, at the same time also display a protective effect in respect of the crop plants.

The weight ratios of herbicide (mixture) to safener generally depend on the application rate of herbicide and the effectiveness of the particular safener and can vary within wide limits, for example in the range from 200:1 to 1:200, preferably 100:1 to 1:100, in particular 20:1 to 1:20. The safeners can be formulated analogously to the compounds of the formula (I) or mixtures thereof with further herbicides/pesticides and can be provided and applied as ready mix or tank mix with the herbicides.

For use, the formulations present in standard commercial form are, if appropriate, diluted in the usual manner, e.g. in the case of wettable powders, emulsifiable concentrates, dispersions and water-dispersible granules by means of water. Dust-like preparations, soil and scatter granules, and also sprayable solutions are usually not diluted any further with further inert substances prior to use.

The required application rate of the compounds of the formula (I) varies inter alia with the external conditions such as temperature, humidity, the type of herbicide used. It can fluctuate within wide limits, e.g. between 0.001 and 10.0 kg/ha or more of active substance, but is preferably between 0.005 and 5 kg/ha.

The present invention is illustrated in more detail by reference to the examples below, although these do not limit the present invention in any way.

### A. SYNTHESIS EXAMPLES

### 1. N-[(4,6-Dimethylpyrimidin-2-yl)carbamoyl]-2-(2,2,2-trifluoro-1-methylethoxy)-benzenesulfonamide (I-1)

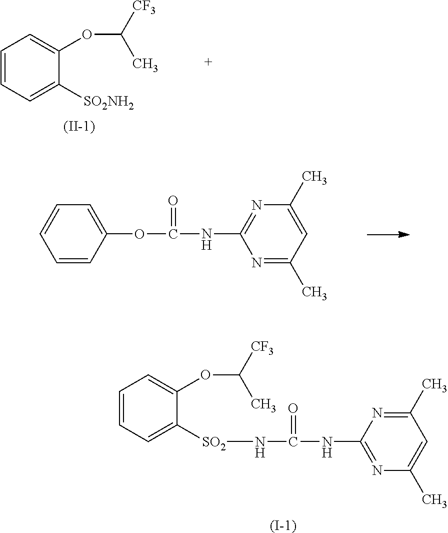

451 mg (1.6 mmol) of 2-(2,2,2-trifluoro-1-methylethoxy)benzenesulfonamide (II-1) are dissolved in 70 ml of acetonitrile, and 270 mg (1.76 mmol) of DBU (diazabicyclo-undecene) are added. 475 mg (1.68 mmol) of phenyl (4,6-dimethylpyrimidin-2-yl)carbamate are added with stirring, and the mixture is stirred for another 16 hours. The mixture is concentrated under reduced pressure and taken up in dichloromethane. The organic phase is washed twice with aqueous hydrochloric acid and then with water and saturated sodium chloride solution. The mixture is concentrated under reduced pressure and taken up in diisopropyl ether. After four days of stirring at 20° C., the white crystals formed are filtered off with suction, washed with diisopropyl ether and dried. This gives 550 mg (1.31 mmol) of N-[(4,6-dimethylpyrimidin-2-yl)carbamoyl]-2-(2,2,2-trifluoro-1-methylethoxy)benzenesulfonamide (I-1) of a purity (HPLC) of 96.8%.

### 2. N-[(4-Methoxy-6-methyl-1,3,5-triazin-2-yl)carbamoyl]-2-(2,2,2-trifluoro-1-methylethoxy)benzenesulfonamide (I-5)

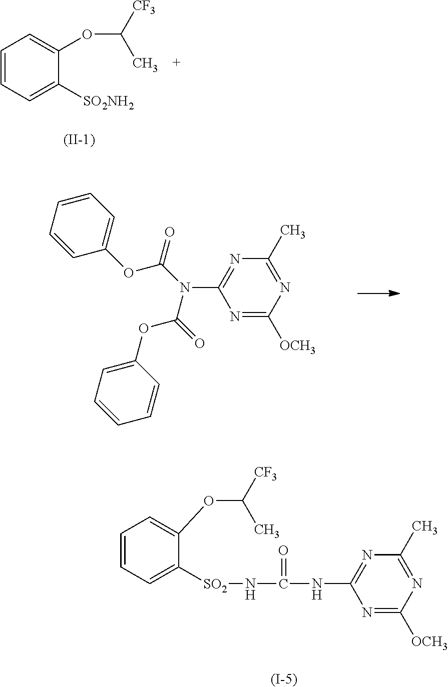

451 mg (1.6 mmol) of 2-(2,2,2-trifluoro-1-methylethoxy)benzenesulfonamide (II-1) are dissolved in 70 ml of acetonitrile, and 270 mg (1.76 mmol) of DBU (diazabicyclo-undecene) are added. 655 mg (1.680 mmol) of diphenyl (4-methoxy-6-methyl-1,3,5-triazin-2-yl)imidodicarbonate (cf. WO 1996/022284) are added with stirring. After 16 hours of stirring at 20° C., the mixture is concentrated under reduced pressure and the residue is taken up in dichloromethane. The organic phase is washed twice with dilute aqueous hydrochloric acid and then washed with water and subsequently dried and concentrated. The oily residue is triturated with diisopropyl ether for 5 days. The crystals formed are filtered off with suction and dried, yield 340 mg, purity according to HPLC 83.5%. The crystals are triturated with a little isopropanol at 20° C. for 16 hours and then filtered off with suction and dried. This gives 180 mg (0.404 mmol) of N-[(4-methoxy-6-methyl-1,3,5-triazin-2-yl)carbamoyl]-2-(2,2,2-trifluoro-1-methylethoxy)benzenesulfonamide of a purity (HPLC) of 97.8%.

The compounds of the formula (I) described in the tables below are obtained according to or analogously to the synthesis examples described above:

TABLE 1
Compounds of the formula (I)
(I)

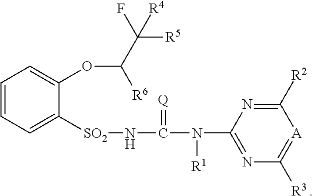

| Ex. | | | | | | | | | |
|---|---|---|---|---|---|---|---|---|---|
| No. | Phys. data | R¹ | R² | R³ | R⁴ | R⁵ | R⁶ | Q | A |
| I-1 | ¹H-NMR | H | CH₃ | CH₃ | F | F | CH₃ | O | CH |
| I-2 | ¹H-NMR | H | CH₃ | OCH₃ | F | F | CH₃ | O | CH |
| I-3 | ¹H-NMR | H | Cl | OCH₃ | F | F | CH₃ | O | CH |
| I-4 | ¹H-NMR | H | OCH₃ | OCH₃ | F | F | CH₃ | O | CH |
| I-5 | ¹H-NMR | H | CH₃ | OCH₃ | F | F | CH₃ | O | N |
| I-6 | ¹H-NMR | H | OCH₃ | OCH₃ | F | F | CH₃ | O | N |
| I-7 | | CH₃ | CH₃ | OCH₃ | F | F | CH₃ | O | N |
| I-8 | | H | N(CH₃)₂ | OCH₂CF₃ | F | F | CH₃ | O | N |
| I-9 | | H | SCH₃ | OCH₃ | F | F | CH₃ | O | CH |
| I-10 | | H | H | OCH₃ | F | F | CH₃ | O | CH |
| I-11 | | H | H | CH₃ | F | F | CH₃ | O | CH |
| I-12 | | H | OCHF₂ | OCHF₂ | F | F | CH₃ | O | CH |
| I-13 | | H | OCH₃ | CHF₂ | F | F | CH₃ | O | N |
| I-14 | | H | CH₃ | CH₃ | F | F | CH₃ | O | N |
| I-15 | | H | CH₃ | CF₃ | F | F | CH₃ | O | N |
| I-16 | | H | CH₃ | OCF₃ | F | F | CH₃ | O | N |
| I-17 | | H | CF₃ | OCH₃ | F | F | CH₃ | O | N |
| I-18 | | H | NHCH₃ | OC₂H₅ | F | F | CH₃ | O | N |
| I-19 | | H | CH₃ | OCH₃ | F | F | CH₃ | S | N |
| I-20 | | H | OCH₃ | OCH₃ | F | F | CH₃ | S | N |
| I-21 | | CH₃ | CH₃ | OCH₃ | F | F | CH₃ | S | N |
| I-22 | ¹H-NMR | H | CH₃ | OCH₃ | F | F | Et | O | CH |
| I-23 | ¹H-NMR | H | CH₃ | OCH₃ | F | F | Et | O | N |
| I-24 | | CH₃ | CH₃ | OCH₃ | F | F | Et | O | N |
| I-25 | ¹H-NMR | H | OCH₃ | OCH₃ | F | F | Et | O | CH |
| I-26 | | H | OCH₃ | OCH₃ | F | F | Et | O | N |
| I-27 | ¹H-NMR | H | OCH₃ | CF₃ | F | F | Et | O | N |
| I-28 | ¹H-NMR | H | OCH₃ | Cl | F | F | Et | O | CH |
| I-29 | ¹H-NMR | H | CH₃ | CH₃ | F | F | Et | O | CH |
| I-30 | | H | CH₃ | OCH₃ | F | F | Pr | O | CH |
| I-31 | | H | CH₃ | OCH₃ | F | F | Pr | O | N |
| I-32 | | CH₃ | CH₃ | OCH₃ | F | F | Pr | O | N |
| I-33 | | H | OCH₃ | OCH₃ | F | F | Pr | O | CH |
| I-34 | | H | OCH₃ | OCH₃ | F | F | Pr | O | N |
| I-35 | | H | CH₃ | OCH₃ | F | F | Allyl | O | CH |
| I-36 | | H | CH₃ | OCH₃ | F | F | Allyl | O | N |
| I-37 | | CH₃ | CH₃ | OCH₃ | F | F | Allyl | O | N |
| I-38 | | H | OCH₃ | OCH₃ | F | F | Allyl | O | CH |
| I-39 | | H | OCH₃ | OCH₃ | F | F | Allyl | O | N |
| I-40 | ¹H-NMR | H | CH₃ | OCH₃ | H | H | CH₂F | O | CH |
| I-41 | ¹H-NMR | H | CH₃ | OCH₃ | H | H | CH₂F | O | N |
| I-42 | | CH₃ | CH₃ | OCH₃ | H | H | CH₂F | O | N |
| I-43 | ¹H-NMR | H | OCH₃ | OCH₃ | H | H | CH₂F | O | CH |
| I-44 | | H | OCH₃ | OCH₃ | H | H | CH₂F | O | N |
| I-45 | ¹H-NMR | H | OCH₃ | CF₃ | H | H | CH₂F | O | N |
| I-46 | ¹H-NMR | H | CH₃ | CH₃ | H | H | CH₂F | O | CH |
| I-47 | ¹H-NMR | H | OCH₃ | Cl | H | H | CH₂F | O | CH |
| I-48 | | H | CH₃ | OCH₃ | H | H | CH₂Cl | O | CH |
| I-49 | | H | CH₃ | OCH₃ | H | H | CH₂Cl | O | N |
| I-50 | | CH₃ | CH₃ | OCH₃ | H | H | CH₂Cl | O | N |
| I-51 | | H | OCH₃ | OCH₃ | H | H | CH₂Cl | O | CH |
| I-52 | | H | OCH₃ | OCH₃ | H | H | CH₂Cl | O | N |
| I-53 | | H | CH₃ | OCH₃ | F | CHF₂ | CH₃ | O | CH |
| I-54 | | H | CH₃ | OCH₃ | F | CHF₂ | CH₃ | O | N |
| I-55 | | CH₃ | CH₃ | OCH₃ | F | CHF₂ | CH₃ | O | N |
| I-56 | | H | OCH₃ | OCH₃ | F | CHF₂ | CH₃ | O | CH |
| I-57 | | H | OCH₃ | OCH₃ | F | CHF₂ | CH₃ | O | N |
| I-58 | | H | CH₃ | OCH₃ | F | CF₃ | Et | O | CH |
| I-59 | | H | CH₃ | OCH₃ | F | CF₃ | Et | O | N |
| I-60 | | CH₃ | CH₃ | OCH₃ | F | CF₃ | Et | O | N |
| I-61 | | H | OCH₃ | OCH₃ | F | CF₃ | Et | O | CH |
| I-62 | | H | OCH₃ | OCH₃ | F | CF₃ | Et | O | N |
| I-63 | | H | CH₃ | OCH₃ | H | H | CH₃ | O | CH |
| I-64 | | H | CH₃ | OCH₃ | H | H | CH₃ | O | N |
| I-65 | | CH₃ | CH₃ | OCH₃ | H | H | CH₃ | O | N |
| I-66 | | H | OCH₃ | OCH₃ | H | H | CH₃ | O | CH |
| I-67 | | H | OCH₃ | OCH₃ | H | H | CH₃ | O | N |
| I-68 | | H | CH₃ | OCH₃ | F | F | CF₃ | O | CH |
| I-69 | | H | CH₃ | OCH₃ | F | F | CF₃ | O | N |
| I-70 | | CH₃ | CH₃ | OCH₃ | F | F | CF₃ | O | N |
| I-71 | | H | OCH₃ | OCH₃ | F | F | CF₃ | O | CH |
| I-72 | | H | OCH₃ | OCH₃ | F | F | CF₃ | O | N |
| I-73 | | H | CH₃ | OCH₃ | F | H | CH₃ | O | CH |
| I-74 | | H | CH₃ | OCH₃ | F | H | CH₃ | O | N |
| I-75 | | CH₃ | CH₃ | OCH₃ | F | H | CH₃ | O | N |
| I-76 | | H | OCH₃ | OCH₃ | F | H | CH₃ | O | CH |
| I-77 | | H | OCH₃ | OCH₃ | F | H | CH₃ | O | N |
| I-78 | | H | CH₃ | OCH₃ | F | CF₃ | CH₃ | O | CH |
| I-79 | ¹H-NMR | H | CH₃ | OCH₃ | F | CF₃ | CH₃ | O | N |
| I-80 | | CH₃ | CH₃ | OCH₃ | F | CF₃ | CH₃ | O | N |
| I-81 | ¹H-NMR | H | OCH₃ | OCH₃ | F | CF₃ | CH₃ | O | CH |
| I-82 | | H | OCH₃ | OCH₃ | F | CF₃ | CH₃ | O | N |
| I-83 | ¹H-NMR | H | OEt | CH₃ | F | CF₃ | CH₃ | O | N |
| I-84 | ¹H-NMR | H | OCH₃ | CF₃ | F | CF₃ | CH₃ | O | N |

¹H-NMR data (400 MHz, solvent: CDCl₃, CD₃CN or [D₆]-DMSO, internal standard: tetramethylsilane δ=0.00 ppm; s=singlet, br. s=broad singlet, d=doublet, dd=doublet of doublets, ddd=doublet of a doublet of doublets, m=multiplet, q=quartet, qnt=quintet, sxt=sextet, spt=septet, t=triplet)

I-1:

¹H-NMR (CD₃CN): δ=1.39 (d, 3H); 2.41 (s, 6H); 5.11 (m, 1H); 6.88 (s, 1H); 7.21 (m, 2H); 7.66 (m, 1H); 7.94 (br. s, 1H); 8.03 (d, 1H); 12.97 (br. s, 1H) ppm

I-2:

¹H-NMR (CD₃CN): δ=1.40 (d, 3H); 2.38 (s, 3H); 3.93 (s, 3H); 5.12 (m, 1H); 6.38 (s, 1H); 7.20 (m, 2H); 7.66 (m, 1H); 7.95 (br. s, 1H); 8.04 (d, 1H); 13.01 (br. s, 1H) ppm

I-3:

¹H-NMR (CD₃CN): δ=1.44 (d, 3H); 4.00 (s, 3H); 6.60 (s, 1H); 7.22 (m, 2H); 7.67 (m, 1H); 8.04 (d, 1H); 8.25 (br. s, 1H); 11.97 (br. s, 1H) ppm

I-4:

¹H-NMR (CD₃CN): δ=1.40 (d, 3H); 3.94 (s, 6H); 5.14 (m, 1H); 5.84 (s, 1H); 7.21 (m, 2H); 7.66 (m, 1H); 8.02 (br. s, 1H); 8.04 (d, 1H); 12.56 (br. s, 1H) ppm

I-5:

¹H-NMR (CD₃CN): δ=1.43 (d, 3H); 2.48 (s, 3H); 4.00 (s, 3H); 5.13 (m, 1H); 7.22 (m, 2H); 7.67 (m, 1H); 8.03 (d, 1H); 8.27 (br. s, 1H); 12.38 (br. s, 1H) ppm

I-6:

¹H-NMR ([D₆]-DMSO): δ=1.37 (d, 3H); 3.99 (s, 6H); 5.48 (m, 1H); 7.24 (t, 1H); 7.44 (d, 1H); 7.70 (dt, 1H); 7.95 (dd, 1H); 10.75 (br. s, 1H); 12.20 (br. s, 1H) ppm

I-22:

¹H-NMR (CDCl₃): δ=12.98 (br. s, 1H); 8.21 (dd, J=1.6, 8.2, 1H); 7.58 (ddd, J=1.6, 7.5, 8.2, 1H); 7.18 (t, J=7.5, 1H); 7.12 (br. s, 1H); 7.00 (d, J=8.5, 1H); 6.29 (s, 1H); 4.69 (sxt, J=6.2, 1H); 3.94 (s, 3H); 2.42 (s, 3H); 1.90 (m, 2H); 1.03 (t, J=7.5, 3H) ppm

I-23:

¹H-NMR (CDCl₃): δ=12.32 (br. s, 1H); 8.19 (dd, J=2.0, 8.2, 1H); 7.60 (ddd, J=2.0, 7.5, 8.8, 1H); 7.23 (br. s, 1H); 7.20 (td, J=7.8, 0.7, 1H); 7.02 (d, J=8.5, 1H); 4.71 (sxt, J=6.2, 1H); 4.05 (s, 3H); 2.57 (s, 3H); 1.95 (m, 2H); 1.07 (t, J=7.5, 3H) ppm

I-25:

¹H-NMR (CDCl₃): δ=12.60 (br. s, 1H); 8.21 (dd, J=2.0, 7.8, 1H); 7.59 (ddd, J=1.6 7.5, 8.5, 1H); 7.19 (td, J=8.2, 1.0, 1H); 7.12 (br. s, 1H); 7.01 (d, J=8.5, 1H); 5.75 (s, 1H); 4.71 (m, 1H); 3.96 (s, 6H); 1.93 (m, 1H); 1.83 (m, 1H); 1.02 (t, J=7.5, 3H) ppm

I-27:

¹H-NMR (CDCl₃): δ=11.52 (br. s, 1H); 8.18 (dd, J=1.6, 7.8, 1H); 7.62 (ddd, J=1.6, 7.5, 8.5, 1H); 7.56 (br. s, 1H); 7.21 (td, J=7.8, 0.7, 1H); 7.04 (d, J=8.5, 1H); 4.71 (sxt, J=6.2, 1H); 4.18 (s, 3H); 1.96 (qnt, J=7.2, 2H); 1.08 (t, J=7.5, 3H) ppm

I-28:

¹H-NMR (CDCl₃): δ=12.00 (br. s, 1H); 8.20 (dd, J=1.6, 7.8, 1H); 7.60 (ddd, J=1.6, 7.5, 8.2, 1H); 7.24 (br. s, 1H); 7.19 (td, J=8.5, 0.7, 1H); 7.02 (d, J=8.5, 1H); 6.49 (s, 1H); 4.70 (sxt, J=6.2, 1H); 4.01 (s, 3H); 1.95 (m, 2H); 1.06 (t, J=7.5, 3H) ppm

I-29:

¹H-NMR (CDCl₃): δ=12.87 (br. s, 1H); 8.21 (dd, J=2.0, 8.2, 1H); 7.58 (ddd, J=1.6, 7.5, 8.5, 1H); 7.29 (br. s, 1H); 7.18 (td, J=8.2, 1.0, 1H); 7.00 (d, J=8.5, 1H); 6.75 (s, 1 h); 4.69 (sxt, J=6.2, 1H); 2.45 (s, 6H); 1.89 (m, 2H); 1.03 (t, J=7.5, 3H) ppm

I-40:

¹H-NMR (CDCl₃): δ=12.92 (br. s, 1H); 8.18 (dd, J=1.6, 7.9, 1H); 7.59 (td, J=8.5, 1.9, 1H); 7.19 (t, J=7.9, 1H); 7.17 (br. s, 1H); 7.09 (d, J=8.3, 1H); 6.29 (s, 1H); 4.81 (m, 1H); 4.72 (m, 2H); 4.60 (m, 2H); 3.93 (s, 3H); 2.43 (s, 3H) ppm

I-41:

¹H-NMR (CDCl₃): δ=12.25 (br. s, 1H); 8.16 (d, J=7.8, 1H); 7.60 (t, J=8.2, 1H); 7.33 (br. s, 1H); 7.19 (t, J=7.5, 1H); 7.10 (d, J=8.5, 1H); 4.79 (m, 3H); 4.63 (m, 1H); 4.05 (s, 3H); 2.57 (s, 3H) ppm

I-43:

¹H-NMR (CDCl₃): δ=12.55 (br. s, 1H); 8.19 (dd, J=1.9, 7.6, 1H); 7.59 (td, J=7.6, 1.9, 1H); 7.19 (t, J=7.0, 1H); 7.15 (br. s, 1H); 7.09 (d, J=8.3, 1H); 5.79 (s, 1H); 4.83 (m, 1H); 4.67 (m, 2H); 4.58 (m, 2H); 3.96 (m, 6H) ppm

I-45:

¹H-NMR (CDCl₃): δ=11.49 (br. s, 1H); 8.16 (dd, J=1.6, 7.8, 1H); 7.63 (m, 3H); 7.21 (t, J=7.8, 1H); 7.10 (d, J=8.2, 1H); 4.86 (m, 1H); 4.73 (m, 2H); 4.61 (m, 2H); 4.18 (s, 3H) ppm

I-46: ¹H-NMR (CDCl₃): δ=12.83 (br. s, 1H); 8.18 (dd, J=2.0, 8.2, 1H); 7.59 (ddd, J=1.6, 7.5, 8.2, 1H); 7.39 (br. s, 1H); 7.19 (td, J=8.2, 0.7, 1H); 7.09 (d, J=8.5, 1H); 6.76 (s, 1H); 4.80 (m, 1H); 4.72 (m, 2H); 4.60 (m, 2H); 2.46 (m, 6H) ppm

I-47:

¹H-NMR (CDCl₃): δ=11.94 (br. s, 1H); 8.17 (dd, J=1.6, 7.8, 1H); 7.61 (td, J=8.8, 1.6, 1H); 7.29 (br. s, 1H); 7.20 (t, J=7.2, 1H); 7.10 (d, J=8.5, 1H); 6.49 (s, 1H); 4.85 (m, 1H); 4.74 (m, 2H); 4.62 (m, 2H); 4.00 (s, 3H) ppm

I-79:

¹H-NMR (CDCl₃): δ=12.29 (br s, 1H); 8.20 (dd, J=1.6, 8.2, 1H); 7.62 (ddd, J=1.6, 7.2, 8.2, 1H); 7.24 (br s, 1H); 7.23 (td, J=8.5, 1.0, 1H); 7.03 (d, J=8.5, 1H); 5.01 (m, 1H); 4.05 (s, 3H); 2.57 (s, 3H); 1.52 (d, J=6.5, 3H) ppm

I-81:

¹H-NMR (CDCl₃): δ=12.53 (br s, 1H); 8.22 (dd, J=1.6, 7.8, 1H); 7.61 (ddd, J=1.6, 7.5, 8.5, 1H); 7.23 (td, J=7.8, 1.0, 1H); 7.14 (br s, 1H); 7.03 (d, J=8.5, 1H); 5.79 (s, 1H); 5.02 (m, 1H); 3.96 (s, 6H); 1.48 (d, J=6.2, 3H) ppm

I-83:

¹H-NMR (CDCl₃): δ=12.33 (br s, 1H); 8.20 (dd, J=2.0, 8.2, 1H); 7.62 (ddd, J=2.0, 7.5, 8.5, 1H); 7.26 (br s, 1H); 7.23 (td, J=8.5, 1.0, 1H); 7.03 (d, J=8.2, 1H); 5.01 (m, 1H); 4.47 (q, J=7.2, 2H); 2.56 (s, 3H); 1.52 (d, J=6.5, 3H); 1.43 (t, J=7.2, 3H) ppm

I-84:

¹H-NMR (CDCl₃): δ=11.49 (br s, 1H); 8.19 (dd, J=1.6, 7.8, 1H); 7.64 (ddd, J=2.0, 7.5, 8.5, 1H); 7.26 (br s, 1H); 7.24 (td, J=8.2, 1.0, 1H); 7.05 (d, J=8.5, 1H); 5.01 (m, 1H); 4.18 (s, 3H); 1.54 (d, J=6.5, 3H) ppm

TABLE 2
Optically active compounds of the formula (I)
(IR)

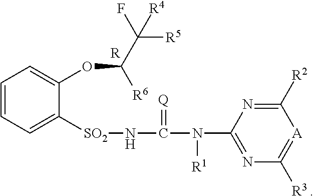

| Ex. | Phys. | | | | | | | | |
|---|---|---|---|---|---|---|---|---|---|
| No. | data | R¹ | R² | R³ | R⁴ | R⁵ | R⁶ | Q | A |
| IR-1 | | H | CH₃ | OCH₃ | F | F | CH₃ | O | CH |
| IR-2 | ¹H-NMR | H | CH₃ | OCH₃ | F | F | CH₃ | O | N |
| IR-3 | | CH₃ | CH₃ | OCH₃ | F | F | CH₃ | O | N |
| IR-4 | ¹H-NMR | H | OCH₃ | OCH₃ | F | F | CH₃ | O | CH |
| IR-5 | | H | OCH₃ | OCH₃ | F | F | CH₃ | O | N |
| IR-6 | | H | CH₃ | OCH₃ | H | H | CH₂Cl | O | CH |
| IR-7 | | H | CH₃ | OCH₃ | H | H | CH₂Cl | O | N |
| IR-8 | | CH₃ | CH₃ | OCH₃ | H | H | CH₂Cl | O | N |
| IR-9 | | H | OCH₃ | OCH₃ | H | H | CH₂Cl | O | CH |
| IR-10 | | H | OCH₃ | OCH₃ | H | H | CH₂Cl | O | N |
| IR-11 | | H | CH₃ | OCH₃ | F | CF₃ | CH₃ | O | CH |
| IR-12 | ¹H-NMR | H | CH₃ | OCH₃ | F | CF₃ | CH₃ | O | N |
| IR-13 | | CH₃ | CH₃ | OCH₃ | F | CF₃ | CH₃ | O | N |
| IR-14 | ¹H-NMR | H | OCH₃ | OCH₃ | F | CF₃ | CH₃ | O | CH |
| IR-15 | | H | OCH₃ | OCH₃ | F | CF₃ | CH₃ | O | N |

IR-2:

¹H-NMR (CDCl₃): δ=12.34 (br. s, 1H); 8.19 (dd, J=1.6, 7.8, 1H); 7.61 (td, J=9.1, 2.0, 1H); 7.25 (br. s, 1H); 7.22 (td, J=8.2, 0.7, 1H); 7.01 (d, J=8.5, 1H); 4.84 (spt, J=6.2, 1H); 4.05 (s, 3H); 2.58 (s, 3H); 1.55 (d, J=6.5, 3H) ppm

IR-4:

¹H-NMR (CDCl₃): δ=12.59 (br. s, 1H); 8.21 (m, 1H); 7.60 (m, 1H); 7.21 (br. t, J=7.8, 1H); 7.12 (br. s, 1H); 7.00 (d, J=8.5, 1H); 5.79 (s, 1H); 4.85 (spt, J=6.2, 1H); 3.96 (s, 6H); 1.50 (d, J=6.2, 3H) ppm

IR-12:

¹H-NMR (CDCl₃): δ=12.29 (br s, 1H); 8.20 (dd, J=1.6, 7.8, 1H); 7.62 (ddd, J=1.6, 7.5, 8.5, 1H); 7.29 (br s, 1H); 7.23 (td, J=8.2, 1.0, 1H); 7.03 (d, J=8.5, 1H); 5.01 (m, 1H); 4.05 (s, 3H); 2.57 (s, 3H); 1.52 (d, J=6.5, 3H) ppm

IR-14:

¹H-NMR (CDCl₃): δ=12.52 (br s, 1H); 8.22 (dd, J=2.0, 8.2, 1H); 7.61 (ddd, J=2.0, 7.5, 8.5, 1H); 7.22 (td, J=8.5, 1.0, 1H); 7.12 (br s, 1H); 7.03 (d, J=8.5, 1H); 5.79 (s, 1H); 5.02 (m, 1H); 3.96 (s, 6H); 1.48 (d, J=6.5, 3H) ppm

TABLE 3
Optically active compounds of the formula (I)
(IS)

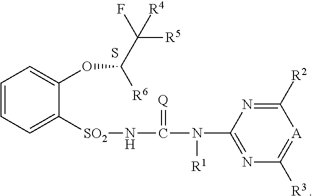

| Ex. | Phys. | | | | | | | | |
|---|---|---|---|---|---|---|---|---|---|
| No. | data | R¹ | R² | R³ | R⁴ | R⁵ | R⁶ | Q | A |
| IS-1 | | H | CH₃ | OCH₃ | F | F | CH₃ | O | CH |
| IS-2 | ¹H-NMR | H | CH₃ | OCH₃ | F | F | CH₃ | O | N |
| IS-3 | | CH₃ | CH₃ | OCH₃ | F | F | CH₃ | O | N |
| IS-4 | ¹H-NMR | H | OCH₃ | OCH₃ | F | F | CH₃ | O | CH |
| IS-5 | ¹H-NMR | H | OCH₃ | OCH₃ | F | F | CH₃ | O | N |
| IS-6 | ¹H-NMR | H | OCH₃ | CF₃ | F | F | CH₃ | O | N |
| IS-7 | | H | CH₃ | OCH₃ | H | H | CH₂Cl | O | CH |
| IS-8 | | H | CH₃ | OCH₃ | H | H | CH₂Cl | O | N |
| IS-9 | | CH₃ | CH₃ | OCH₃ | H | H | CH₂Cl | O | N |
| IS-10 | | H | OCH₃ | OCH₃ | H | H | CH₂Cl | O | CH |
| IS-11 | | H | OCH₃ | OCH₃ | H | H | CH₂Cl | O | N |
| IS-12 | | H | CH₃ | OCH₃ | F | CF₃ | CH₃ | O | CH |
| IS-13 | ¹H-NMR | H | CH₃ | OCH₃ | F | CF₃ | CH₃ | O | N |
| IS-14 | | CH₃ | CH₃ | OCH₃ | F | CF₃ | CH₃ | O | N |
| IS-15 | ¹H-NMR | H | OCH₃ | OCH₃ | F | CF₃ | CH₃ | O | CH |
| IS-16 | ¹H-NMR | H | OCH₃ | OCH₃ | F | CF₃ | CH₃ | O | N |

IS-2:

¹H-NMR (CDCl₃): δ=12.33 (br s, 1H); 8.19 (br d, J=8.2, 1H); 7.61 (br t, J=7.5, 1H); 7.29 (br s, 1H); 7.21 (t, J=7.8, 1H); 7.02 (d, J=8.5, 1H); 4.85 (m, 1H); 4.05 (s, 3H); 2.58 (s, 3H); 1.55 (d, J=6.5, 3H) ppm

IS-4:

¹H-NMR (CDCl₃): δ=12.60 (br. s, 1H); 8.21 (dd, J=1.6, 7.8, 1H); 7.61 (td, J=7.5, 1.6, 1H); 7.21 (t, J=7.5, 1H); 7.13 (br. s, 1H); 7.00 (d, J=8.5, 1H); 5.79 (s, 1H); 4.85 (spt, J=6.2, 1H); 3.96 (s, 6H); 1.50 (d, J=6.5, 3H) ppm

IS-5:

¹H-NMR (CDCl₃): δ=12.18 (br s, 1H); 8.18 (dd, J=1.3, 7.8, 1H); 7.61 (br t, J=8.5, 1H); 7.26 (br s, 1H); 7.21 (t, J=7.8, 1H); 7.01 (d, J=8.5, 1H); 4.84 (m, 1H); 4.06 (s, 6H); 1.56 (d, J=6.5, 3H) ppm

IS-6:

¹H-NMR (CDCl₃): δ=11.54 (br. s, 1H); 8.18 (dd, J=1.6, 7.8, 1H); 7.64 (m, 1H); 7.62 (br. s, 1H); 7.23 (t, J=7.5, 1H); 7.04 (d, J=8.5, 1H); 4.85 (spt, J=6.2, 1H); 4.18 (s, 3H); 1.57 (d, J=6.2, 3H) ppm

IS-13:

¹H-NMR (CDCl₃): δ=12.30 (br s, 1H); 8.20 (dd, J=1.6, 7.8, 1H); 7.62 (ddd, J=2.0, 7.5, 8.5, 1H); 7.26 (br s, 1H); 7.23 (td, J=7.8, 1.0, 1H); 7.03 (d, J=8.5, 1H); 5.02 (m, 1H); 4.05 (s, 3H); 2.57 (s, 3H); 1.52 (d, J=6.5, 3H) ppm

IS-15:

¹H-NMR (CDCl₃): δ=12.52 (br s, 1H); 8.22 (dd, J=1.6, 7.8, 1H); 7.61 (ddd, J=2.0, 7.5, 8.5, 1H); 7.22 (td, J=7.8, 1.0, 1H); 7.12 (br s, 1H); 7.03 (d, J=8.5, 1H); 5.79 (s, 1H); 5.02 (m, 1H); 3.96 (s, 6H); 1.48 (d, J=6.5, 3H) ppm

IS-16:

¹H-NMR (CDCl₃): δ=12.13 (br s, 1H); 8.20 (dd, J=2.0, 8.2, 1H); 7.62 (ddd, J=1.6, 7.5, 8.5, 1H); 7.22 (br s, 1H); 7.22 (td, J=8.5, 1.0, 1H); 7.04 (d, J=8.5, 1H); 5.01 (m, 1H); 4.06 (s, 6H); 1.54 (d, J=6.5, 3H) ppm

### 3. 2-(2,2,2-Trifluoro-1-methylethoxy)benzenesulfonamide (II-1)

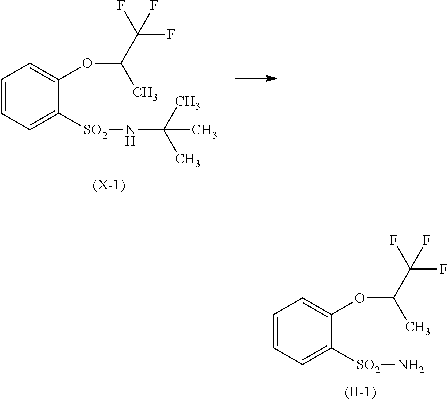

42.5 g of N-tert-butyl-2-(2,2,2-trifluoro-1-methylethoxy)benzenesulfonamide (0.0948 mol, purity according to HPLC: 72.6%) are dissolved in 250 ml of ethyl acetate, and 432 g (3.79 mol) of trifluoroacetic acid are added, which results in slight warming. The dark-brown solution is allowed to stand at 20° C. for 16 hours, concentrated under reduced pressure, once more taken up in trifluoroacetic acid and concentrated again. The residue is taken up in dichloromethane and washed repeatedly with saturated sodium bicarbonate solution until the washings are no longer acidic. The organic phase is dried, filtered through Celite and concentrated. This gives 32 g of crude material which, according to thin-layer chromatography (dichloromethane) is slightly contaminated. For further purification, the material is chromatographed on 370 g of silica gel 60. Concentration of the organic phase gives 20.9 g (0.717 mol, purity according to HPLC: 92.4%) of light-yellow crystals. The crystals are triturated with petroleum ether/ether (about 10/1). This gives 19.4 g (0.0705 mol) of light-yellow crystals (purity according to HPLC: 97.9%).

The following compounds can be prepared analogously:

TABLE 4
Compounds of the formula (II)
(II)

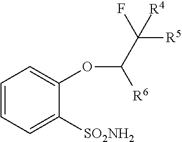

| Ex. No. | Phys. data | R⁴ | R⁵ | R⁶ |
|---|---|---|---|---|
| II-1 | ¹H-NMR | F | F | CH₃ |
| II-2 | ¹H-NMR | F | F | C₂H₅ |
| II-3 | | F | F | CF₃ |
| II-4 | | F | F | C₃H₇-n |
| II-5 | | F | F | Allyl |
| II-6 | ¹H-NMR | H | H | CH₂F |
| II-7 | | H | H | CH₂Cl |
| II-8 | | F | CHF₂ | CH₃ |
| II-9 | | F | CF₃ | C₂H₅ |
| II-10 | | H | H | CH₃ |
| II-11 | | H | F | CH₃ |
| II-12 | ¹H-NMR | F | CF₃ | CH₃ |

II-1:

¹H-NMR ([D₆]-DMSO): δ=1.48 (d, 3H); 5.42 (m, 1H); 6.75 (br. s, 2H); 7.16 (t, 1H); 7.39 (d, 1H); 7.58 (dt, 1H); 7.81 (dd, 1H) ppm

II-2:

¹H-NMR (CDCl₃): δ=7.97 (dd, J=1.6, 7.6, 1H); 7.55 (ddd, J=1.9, 7.6, 8.6, 1H); 7.14 (td, J=7.6, 1.0, 1H); 7.07 (d, J=8.3, 1H); 4.99 (br s, 2H); 4.79 (sxt, J=6.4, 1H); 2.04 (qnt, J=7.0, 2H); 1.12 (t, J=7.0, 3H) ppm

II-6:

¹H-NMR (CDCl₃): δ=7.93 (dd, J=1.3, 7.6, 1H); 7.56 (ddd, J=1.6, 7.6, 7.9, 1H); 7.15 (m, 2H); 5.11 (br. s, 2H); 4.86 (m, 3H); 4.73 (m, 2H) ppm

II-12:

¹H-NMR (CDCl₃): δ=7.96 (dd, J=1.3, 7.6, 1H); 7.59 (td, J=7.6, 1.9, 1H); 7.19 (t, J=7.6, 1H); 7.09 (d, J=8.3, 1H); 5.30 (br s, 1H); 5.09 (m, 1H); 5.06 (br s, 1H); 1.58 (d, J=6.4, 3H) ppm

TABLE 5
Optically active compounds of the formula (II)
(IIR)

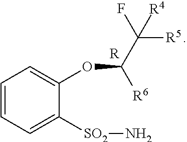

| Ex. No. | Phys. data | R⁴ | R⁵ | R⁶ |
|---|---|---|---|---|
| IIR-1 | ¹H-NMR | F | F | CH₃ |
| IIR-2 | | F | F | C₂H₅ |
| IIR-4 | | F | F | C₃H₇-n |
| IIR-5 | | F | F | Allyl |
| IIR-7 | | H | H | CH₂Cl |
| IIR-8 | | F | CHF₂ | CH₃ |
| IIR-9 | | F | CF₃ | C₂H₅ |
| IIR-10 | | H | H | CH₃ |
| IIR-11 | | H | F | CH₃ |
| IIR-12 | ¹H-NMR | F | CF₃ | CH₃ |

IIR-1:

¹H-NMR (CDCl₃): δ=7.80 (dd, J=1.6, 7.6, 1H); 7.60 (ddd, J=1.6, 7.3, 8.3, 1H); 7.43 (d, J=8.3, 1H); 7.16 (td, J=7.9, 1.0, 1H); 6.95 (br. s, 2H); 5.49 (qnt, J=6.4, 1H); 1.48 (d, J=6.4, 3H) ppm

IIR-12:

¹H-NMR (CDCl₃): δ=7.95 (dd, J=1.9, 7.6, 1H); 7.56 (td, J=8.3, 1.3, 1H); 7.17 (t, J=7.6, 1H); 7.10 (d, J=7.6, 1H); 5.85 (br s, 2H); 5.12 (m, 1H); 1.57 (d, J=6.4, 3H) ppm

TABLE 6
Optically active compounds of the formula (II)
(IIS)

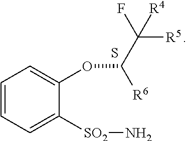

| Ex. No. | Phys. data | R⁴ | R⁵ | R⁶ |
|---|---|---|---|---|
| IIS-1 | ¹H-NMR | F | F | CH₃ |
| IIS-2 | | F | F | C₂H₅ |
| IIS-4 | | F | F | C₃H₇-n |
| IIS-5 | | F | F | Allyl |
| IIS-7 | | H | H | CH₂Cl |
| IIS-8 | | F | CHF₂ | CH₃ |
| IIS-9 | | F | CF₃ | C₂H₅ |
| IIS-10 | | H | H | CH₃ |
| IIS-11 | | H | F | CH₃ |
| IIS-12 | ¹H-NMR | F | CF₃ | CH₃ |

IIS-1:

¹H-NMR (CDCl₃): δ=7.97 (dd, J=1.6, 7.9, 1H); 7.56 (ddd, J=1.9, 7.6, 8.6, 1H); 7.17 (td, J=7.6, 1.0, 1H); 7.07 (d, J=8.3, 1H); 4.98 (br. s, 2H); 4.92 (spt, J=6.4, 1H); 1.61 (d, J=6.4, 3H) ppm

IIS-12:

¹H-NMR (CDCl₃): δ=7.97 (dd, J=1.9, 7.6, 1H); 7.58 (td, J=8.3, 1.9, 1H); 7.18 (td, J=7.6, 1.3, 1H); 7.08 (d, J=8.3, 1H); 5.09 (m, 1H); 5.00 (br s, 2H); 1.58 (d, J=6.4, 3H) ppm

### 4. N-tert-Butyl-2-(2,2,2-trifluoro-1-methylethoxy)benzenesulfonamide (X-1)

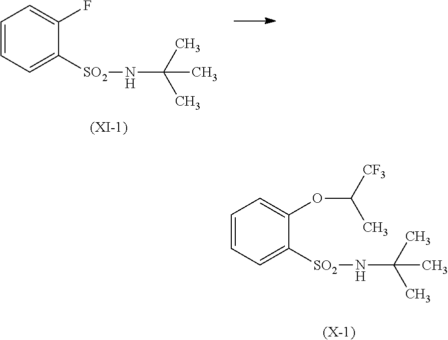

1.01 g (40 mmol) of sodium hydride are initially charged in 35 ml of anhydrous tetrahydrofuran, and 4.656 g (40 mmol) of 1,1,1-trifluoropropan-2-ol are added carefully with stirring. With generation of heat, a clear solution is formed, which is stirred for about 30 min. 3.256 g (13.33 mmol) of N-tert-butyl-2-fluoro-benzenesulfonamide (cf. WO 2006/114220) are added. At 150° C., the reaction mixture is irradiated in a microwave apparatus (CEM Discover model) at 200 W for 1 hour. The cold brown reaction mixture is concentrated under reduced pressure, taken up in dichloromethane and washed with dilute hydrochloric acid and water. Drying and concentration give 4.9 g of a brown oil which, according to HPLC, is about 78% pure and is used without further purification for the next reaction.

Several repeat reactions gave yields of 70-89% of theory with purities of 68-82%.

### 5. N-tert-Butyl-2-[(1,3-difluoropropan-2-yl)oxy]benzenesulfonamide (X-6)

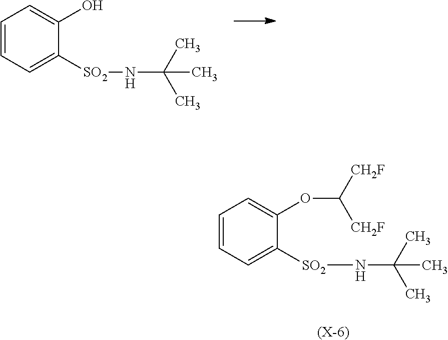

Under a protective argon atmosphere, 4.00 g (17.44 mmol) of N-tert-butyl-2-hydroxybenzenesulfonamide (cf. WO 2000/035442 or EP 574090) are dissolved in 100 ml of anhydrous tetrahydrofuran, and 9.15 g (34.89 mmol) and then 3.35 g (34.89 mmol) of 1,3-difluoropropan-2-ol are added. The reaction solution is cooled to 0° C., and 7.06 g (34.89 mmol) of diisopropyl azodicarboxylate are slowly added dropwise with stirring at this temperature. The reaction solution is slowly warmed to room temperature and then stirred at this temperature for 4 hours. The mixture is then concentrated at 35° C. under reduced pressure. The residue is taken up in ethyl acetate, washed twice with dilute aqueous sodium bicarbonate solution and once with water, dried and concentrated. The residue is purified on a medium-pressure chromatography station (3 bar, silica gel 60, ethyl acetate/n-heptane). This gives 4.2 g (13.66 mmol) of N-tert-butyl-2-[(1,3-difluoropropan-2-yl)oxy]benzenesulfonamide as a colorless solid.

The following compounds can be prepared in an analogous manner:

TABLE 7
Compounds of the formula (X)
(X)

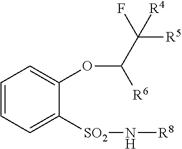

| Ex. No. | Phys. data | R⁴ | R⁵ | R⁶ | R⁸ |
|---|---|---|---|---|---|
| X-1 | ¹H-NMR | F | F | CH₃ | C₄H₉-t |
| X-2 | ¹H-NMR | F | F | C₂H₅ | C₄H₉-t |
| X-3 | | F | F | CF₃ | C₄H₉-t |
| X-4 | | F | F | C₃H₇-n | C₄H₉-t |
| X-5 | | F | F | Allyl | C₄H₉-t |
| X-6 | ¹H-NMR | H | H | CH₂F | C₄H₉-t |
| X-7 | | H | H | CH₂Cl | C₄H₉-t |
| X-8 | | F | CHF₂ | CH₃ | C₄H₉-t |
| X-9 | | F | CF₃ | C₂H₅ | C₄H₉-t |
| X-10 | | H | H | CH₃ | C₄H₉-t |
| X-11 | | H | F | CH₃ | C₄H₉-t |
| X-12 | ¹H-NMR | F | CF³ | CH₃ | C₄H₉-t |

X-1:

¹H-NMR (CD₃CN): δ=1.16 (s, 9H); 1.54 (d, 3H); 4.95 (br. s, 1H); 5.18 (m, 1H); 7.14-7.21 (m, 2H); 7.58 (t, 1H); 7.88 (dd, 1H) ppm

X-2:

¹H-NMR (CDCl₃): δ=7.97 (dd, J=1.9, 7.9, 1H); 7.51 (ddd, J=1.6, 7.3, 8.3, 1H); 7.12 (td, J=7.9, 1.0, 1H); 7.02 (d, J=8.6, 1H); 4.79 (m, 2H); 2.00 (qnt, J=7.9, 2H); 1.19 (s, 9H); 1.10 (t, J=7.6, 3H) ppm

X-6:

¹H-NMR (CDCl₃): δ=7.95 (dd, J=1.6, 7.6, 1H); 7.51 (ddd, J=1.9, 7.6, 8.3, 1H); 7.14 (td, J=7.6, 1.0, 1H); 7.11 (d, J=8.3, 1H); 4.78 (m, 6H); 1.20 (s, 9H) ppm

X-12:

7.98 (dd, J=1.9, 7.6, 1H); 7.53 (td, J=7.0, 1.9, 1H); 7.16 (t, J=7.6, 1H); 7.05 (d, J=7.6, 1H); 5.06 (m, 1H); 4.72 (br s, 1H); 1.55 (d, J=6.4, 3H); 1.19 (s, 9H) ppm

TABLE 8
Optically active compoundsof the formula (X)
(XR)

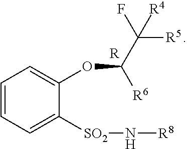

| Ex. No. | Phys. data | R⁴ | R⁵ | R⁶ | R⁸ |
|---|---|---|---|---|---|
| XR-1 | ¹H-NMR | F | F | CH₃ | C₄H₉-t |
| XR-2 | | F | F | C₂H₅ | C₄H₉-t |
| XR-4 | | F | F | C₃H₇-n | C₄H₉-t |
| XR-5 | | F | F | Allyl | C₄H₉-t |
| XR-7 | | H | H | CH₂Cl | C₄H₉-t |
| XR-8 | | F | CHF₂ | CH₃ | C₄H₉-t |
| XR-9 | | F | CF₃ | C₂H₅ | C₄H₉-t |
| XR-10 | | H | H | CH₃ | C₄H₉-t |
| XR-11 | | H | F | CH₃ | C₄H₉-t |
| XR-12 | ¹H-NMR | F | CF₃ | CH₃ | C₄H₉-t |

XR-1:

¹H-NMR (CDCl₃): δ=7.97 (dd, J=1.9, 7.9, 1H); 7.52 (ddd, J=1.9, 7.6, 8.6, 1H); 7.15 (td, J=7.6, 1.0, 1H); 7.02 (d, J=8.3, 1H); 4.93 (spt, J=6.0, 1H); 4.76 (br. s, 1H); 1.58 (d, J=6.7, 3H); 1.19 (s, 9H) ppm

XR-12:

¹H-NMR (CDCl₃): δ=7.98 (dd, J=1.9, 8.3, 1H); 7.53 (td, J=8.3, 1.9, 1H); 7.16 (t, J=7.6, 1H); 7.04 (d, J=7.6, 1H); 5.06 (m, 1H); 4.73 (br s, 1H); 1.55 (d, J=6.4, 3H); 1.19 (s, 9H) ppm

TABLE 9
Optically active compounds of the formula (X)
(XS)

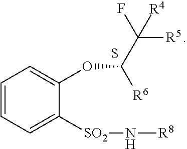

| Ex. No. | Phys. data | R⁴ | R⁵ | R⁶ | R⁸ |
|---|---|---|---|---|---|
| XS-1 | ¹H-NMR | F | F | CH₃ | C₄H₉-t |
| XS-2 | | F | F | C₂H₅ | C₄H₉-t |
| XS-4 | | F | F | C₃H₇-n | C₄H₉-t |
| XS-5 | | F | F | Allyl | C₄H₉-t |
| XS-7 | | H | H | CH₂Cl | C₄H₉-t |
| XS-8 | | F | CHF₂ | CH₃ | C₄H₉-t |
| XS-9 | | F | CF₃ | C₂H₅ | C₄H₉-t |
| XS-10 | | H | H | CH₃ | C₄H₉-t |
| XS-11 | | H | F | CH₃ | C₄H₉-t |
| XS-12 | ¹H-NMR | F | CF₃ | CH₃ | C₄H₉-t |

XS-1:

¹H-NMR (CDCl₃): δ=7.97 (dd, J=1.6, 7.6, 1H); 7.52 (ddd, J=1.6, 7.3, 8.3, 1H); 7.14 (td, J=7.9, 1.0, 1H); 7.02 (d, J=8.3, 1H); 4.93 (spt, J=6.0, 1H); 4.77 (br. s, 1H); 1.58 (d, J=6.7, 3H); 1.19 (s, 9H) ppm

XS-12:

¹H-NMR (CDCl₃): δ=7.98 (dd, J=1.3, 7.6, 1H); 7.53 (td, J=7.6, 1.9, 1H); 7.17 (t, J=7.6, 1H); 7.05 (d, J=8.3, 1H); 5.06 (m, 1H); 4.72 (br s, 1H); 1.55 (d, J=7.0, 3H); 1.19 (s, 9H) ppm

### B. FORMULATION EXAMPLES

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetting agent and dispersant and grinding the mixture in a pinned-disk mill.
c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approximately 255 to above 277° C.) and grinding the mixture in a ball mill to a fineness of below 5 micrometers.
d) An emulsifiable concentrate is obtained from 15 parts by weight of a compound of the formula (I), 75 parts by weight of cyclohexanone as solvent and 10 parts by weight of ethoxylated nonylphenol as emulsifier.
e) Water-dispersible granules are obtained by mixing
   75 parts by weight of a compound of the formula (I),
   10″ of calcium lignosulfonate,
   5″ of sodium lauryl sulfate,
   3″ of polyvinyl alcohol and
   7″ of kaolin,
   grinding the mixture in a pinned-disk mill and granulating the powder in a fluidized bed by spraying on water as granulation liquid.
f) Water-dispersible granules are also obtained by homogenizing and precomminuting, in a colloid mill,
   25 parts by weight of a compound of the formula (I),
   5″ of sodium 2,2′-dinaphthylmethane-6,6′-disulfonate,
   2″ of sodium oleoylmethyltaurinate,
   1 part by weight of polyvinyl alcohol,
   17 parts by weight of calcium carbonate and
   50″ of water,
   then grinding the mixture in a bead mill and atomizing and drying the resulting suspension in a spray tower by means of a single-substance nozzle.

### C. BIOLOGICAL EXAMPLES

1. Pre-Emergence Herbicidal Activity

Seeds of monocotyledonous or dicotyledonous weed plants or crop plants are placed in sandy loam in wood fiber pots and covered with soil. The compounds according to the invention, which are formulated as wettable powders (WP), are then applied to the surface of the soil cover in the form of an aqueous suspension or emulsion with a water application rate of 600 l/ha (converted), with addition of 0.2% wetting agent.

After the treatment, the pots are placed in a greenhouse and maintained under good growth conditions for the test plants. After approximately 3 weeks, the activity of the preparations is scored visually in comparison with untreated controls (herbicidal activity in percent (%): 100% activity=the plants have died, 0% activity=like control plants).

As demonstrated by the results, compounds according to the invention have good herbicidal pre-emergence activity against a broad spectrum of weed grasses and broad-leave weeds. The compounds according to the invention have, for example, very good herbicidal activity against harmful plants such as, for example, Alopecurus myosuroides, Cyperus esculentus, Lolium multiflorum, Matricaria inodora, Pharbitis purpurea, Stellaria media, Veronica persica and Viola tricolor when applied by the pre-emergence method at an application rate of 0.08 kg and less of active substance per hectare.

The following results were achieved when using the compounds according to the invention pre-emergence:

| | Application | | | | | | | | | |
|---|---|---|---|---|---|---|---|---|---|---|
| Compound | Rate | Unit | ALOMY | CYPES | LOLMU | MATIN | PHBPU | STEME | VERPE | VIOTR |
| I-1 | 80 | g/ha | 80 | | 80 | | | | | |
| I-2 | 80 | g/ha | 90 | 100 | 90 | 80 | 80 | 80 | 80 | 80 |
| I-3 | 80 | g/ha | 80 | 100 | 80 | 80 | 80 | | 80 | |
| I-4 | 80 | g/ha | 80 | 90 | 80 | 80 | 80 | | 80 | 80 |
| I-5 | 80 | g/ha | 90 | | | 80 | 80 | 80 | 90 | 80 |
| I-6 | 80 | g/ha | 80 | 100 | | 80 | 80 | 80 | 80 | 80 |

In the table, the individual crops are abbreviated as follows:
ALOMY: Slender meadow foxtail (Alopecurus myosuroides)
CYPES: Chufa Sedge (Cyperus esculentus)
LOLMU: Italian ryegrass (Lolium multiflorum)
MATIN: Scentless camomile (Matricaria inodora)
PHBPU: Purple morning glory (Pharbitis/Ipomoea purpurea)
STEME: Chickweed (Stellaria media)
VERPE: Persian speedwell (Veronica persica)
VIOTR: Wild pansy (Viola tricolor)

2. Post-Emergence Herbicidal Activity

Seeds of monocotyledonous or dicotyledonous weed plants or crop plants are placed in sandy loam in wood fiber pots, covered with soil and grown in a greenhouse under good growth conditions. 2 to 3 weeks after sowing, the test plants are treated in the one-leaf stage. The compounds according to the invention, which are formulated as wettable powders (WP), are then sprayed onto the green plant parts in the form of an aqueous suspension or emulsion with a water application rate of 600 l/ha (converted) with addition of 0.2% of wetting agent. After the test plants have been left to stand in the greenhouse for approximately 3 weeks under optimal growth conditions, the activity of the preparation is scored visually in comparison with untreated controls (herbicidal activity in percent (%): 100% activity=the plants have died, 0% activity=like control plants).

As demonstrated by the results, compounds according to the invention have good herbicidal post-emergence activity against a broad spectrum of weed grasses and broad-leaved weeds. The compounds according to the invention have, for example, very good herbicidal activity against harmful plants such as, for example, Alopecurus myosuroides, Cyperus esculentus, Echinochloa crus-galli, Abutilon theophrasti, Amaranthus retroflexus, Matricaria inodora, Pharbitis purpurea, Polygonum convolvulus and Viola tricolor when applied by the post-emergence method at an application rate of 0.08 kg and less of active substance per hectare.

The following results were achieved when using the compounds according to the invention post-emergence:

| | Application | | | | | | | | | | |
|---|---|---|---|---|---|---|---|---|---|---|---|
| Compound | Rate | Unit | ALOMY | CYPES | ECHCG | ABUTH | AMARE | MATIN | PHBPU | POLCO | VIOTR |
| I-1 | 80 | g/ha | 90 | 80 | 80 | 90 | 80 | 80 | | 90 | |
| I-2 | 80 | g/ha | 90 | 80 | 80 | 90 | | 80 | 80 | 90 | 90 |
| I-3 | 80 | g/ha | 80 | 80 | | 90 | | 90 | 90 | 80 | 80 |
| I-4 | 80 | g/ha | 90 | 80 | 80 | 100 | | 90 | 80 | 80 | 90 |
| I-5 | 80 | g/ha | 90 | | | 90 | 100 | 90 | 90 | 90 | 90 |
| I-6 | 80 | g/ha | | | | 90 | 80 | 90 | 90 | 90 | 90 |

In the table, the individual crops are abbreviated as follows:
ALOMY: Slender meadow foxtail (Alopecurus agrestis L.)
CYPES: Chufa Sedge (Cyperus esculentus)
ECHCG: Barnyard grass (Echinochloa crus-galli)
ABUTH: Velvetleaf (Abuthilon theophrasti)
AMARE: Redroot pigweed (Amaranthus retroflexus)
MATIN: Scentless camomile (Matricaria inodora)
PHBPU: Purple morning glory (Pharbitis (Ipomoea) purpurea)
POLCO: Black bindweed (Fallopia convolvulus)
VIOTR: Wild pansy (Viola tricolor)

1. An N-azinyl-N′-phenylsulfonylurea of the formula (I) 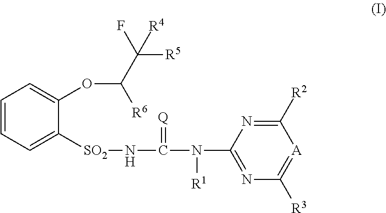
in which
A is selected from the group consisting of nitrogen and CR⁷;
where
R⁷ is selected from the group consisting of hydrogen, alkyl, halogen and haloalkyl;
R¹ is selected from the group consisting of hydrogen and an optionally substituted radical from the group consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted cycloalkyl, optionally halogen-substituted alkoxy, optionally halogen-substituted alkylthio, optionally halogen-substituted alkylamino and optionally halogen-substituted dialkylamino;
R³ is selected from the group consisting of hydrogen, halogen, optionally halogen-substituted alkyl, optionally halogen-substituted cycloalkyl, optionally halogen-substituted alkoxy, optionally halogen-substituted alkylthio, optionally halogen-substituted alkylamino and optionally halogen-substituted dialkylamino;
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl;
Q is selected from the group consisting of oxygen and sulfur;
and/or a salt of a compound of the formula (I).

2. The N-azinyl-N′-phenylsulfonylurea as claimed in claim 1, wherein the substituent A is selected from the group consisting of nitrogen and CH.

3. The N-azinyl-N′-phenylsulfonylurea as claimed in claim 1, wherein the substituent R¹ is selected from the group consisting of hydrogen, optionally halogen-substituted alkyl, optionally halogen-substituted alkoxy, optionally halogen-substituted alkoxyalkyl, optionally halogen-substituted alkenyl and optionally halogen-substituted alkynyl.

4. The N-azinyl-N′-phenylsulfonylurea as claimed in claim 1, wherein the substituent R² is selected from the group consisting of hydrogen, halogen, optionally halogen-substituted alkyl, optionally halogen-substituted cyclopropyl, optionally halogen-substituted alkoxy, optionally halogen-substituted alkylthio, optionally halogen-substituted alkylamino and optionally halogen-substituted dialkylamino.

5. The N-azinyl-N′-phenylsulfonylurea as claimed in claim 1, wherein the substituent R³ is selected from the group consisting of hydrogen, halogen, optionally halogen-substituted alkyl, optionally halogen-substituted cyclopropyl, optionally halogen-substituted alkoxy, optionally halogen-substituted alkylthio, optionally halogen-substituted alkylamino and optionally halogen-substituted dialkylamino.

6. The N-azinyl-N′-phenylsulfonylurea as claimed in claim 1, wherein the substituents R⁴ and R⁵ are each independently of one another selected from the group consisting of hydrogen, fluorine, chlorine, bromine, methyl, difluoromethyl and trifluoromethyl.

7. The N-azinyl-N′-phenylsulfonylurea as claimed in claim 1, wherein the substituent R⁶ is selected from the group consisting of optionally halogen-substituted C₁-C₃-alkyl and C₂-C₄-alkenyl.

8. The N-azinyl-N′-phenylsulfonylurea as claimed in claim 1, wherein the substituents R⁴ and R⁵ are each independently of one another selected from the group consisting of hydrogen, fluorine and trifluoromethyl.

9. The N-azinyl-N′-phenylsulfonylurea as claimed in claim 1, wherein the substituent R⁶ is selected from the group consisting of optionally fluorine- or chlorine-substituted C₁-C₂-alkyl.

10. The N-azinyl-N′-phenylsulfonylurea as claimed in claim 1, wherein the substituents R⁴ and R⁵ are fluorine.

11. The N-azinyl-N′-phenylsulfonylurea as claimed in claim 1, wherein the substituent R⁶ is selected from the group consisting of optionally fluorine-substituted C₁-C₂-alkyl.

12. The N-azinyl-N′-phenylsulfonylurea as claimed in claim 1, wherein ureas are present in a stereochemical purity of more than 50% to 100% in the (R) or (S) configuration with respect to the carbon atom marked by an (*):

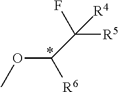

13. A process for preparing an N-azinyl-N′-phenylsulfonylurea as claimed in claim 1, which comprises one of the process steps below:
(a) reaction of 2-(2-fluoroalkoxy)benzenesulfonamide of formula (II) 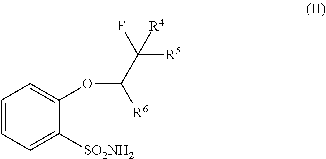
with a heterocyclic (thio)carbamate of formula (III) 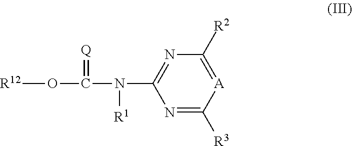
in which R¹² is a substituted or unsubstituted (C₁-C₂₀)-hydrocarbon radical or
(b) reaction of 2-(2-fluoroalkoxy)benzenesulfonyl iso(thio)cyanate of formula (IV) 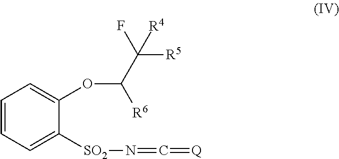
with an aminoheterocycle of formula (V) 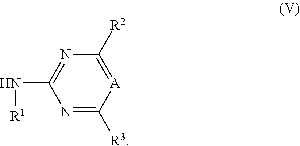
or
(c) reaction of 2-(2-fluoroalkoxy)benzenesulfonyl (thio)carbamate of formula (VI) 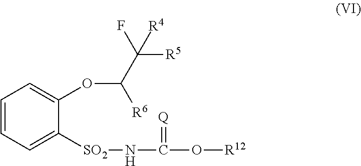
in which R¹² is a substituted or unsubstituted (C₁-C₂₀)-hydrocarbon radical with an aminoheterocycle of formula (V) 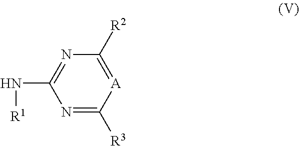
or
(d) reaction of 2-(2-fluoroalkoxy)benzenesulfonamide of formula (II) 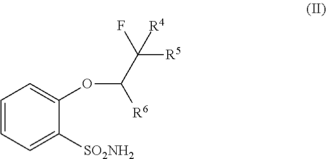
with an iso(thio)cyanate of formula (VII) 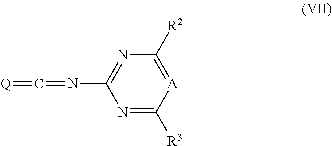
if appropriate in the presence of a reaction auxiliary, where R¹ is hydrogen; or
(e) reaction of an aminoheterocycle of formula (V) 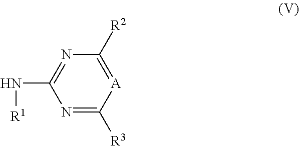
initially under base catalysis with a carbonic ester, and reaction of an intermediate of formula (III) formed 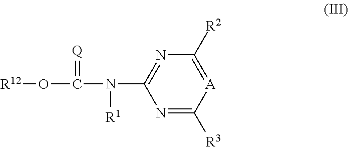
in which R¹² is a substituted or unsubstituted (C₁-C₂₀)-hydrocarbon radical, in a one-pot reaction with a 2-(2-fluoroalkoxy)benzenesulfonamide of the formula (II) 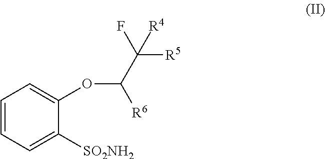
; or
(f) reaction of 2-(2-fluoroalkoxy)benzenesulfonyl halide of formula (VIII) 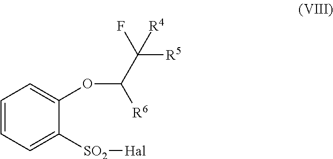
where Hal is a halogen atom, with an alkali metal or alkaline earth metal (thio)cyanate to give a sulfonyl iso(thio)cyanate of formula (IV) 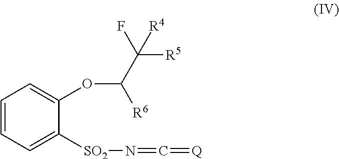
or a solvated (stabilized) derivative thereof, and subsequently with an aminoheterocycle of formula (V) 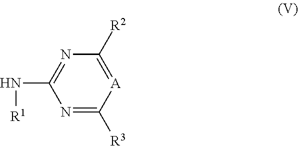
; or
(g) reaction of 2-(2-fluoroalkoxy)benzenesulfonamide of formula (II) with a heterocyclic biscarbamate of the formula (IX), 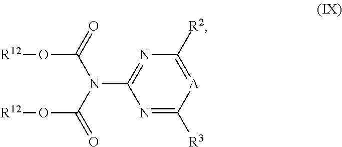
in which R¹² is a substituted or unsubstituted (C₁-C₂₀)-hydrocarbon radical, in the presence of a basic reaction auxiliary, where Q=oxygen and R¹=hydrogen; or
(h) reaction of 2-(2-fluoroalkoxy)benzenesulfonamide of formula (II) 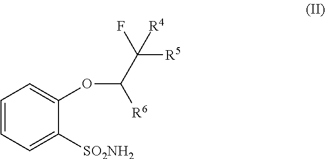
under base catalysis with a carbonic ester and reaction of an intermediate of formula (VI) formed 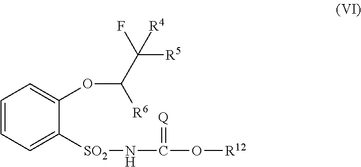
in which R¹² is a substituted or unsubstituted (C₁-C₂₀)-hydrocarbon radical in a one-pot reaction with an aminoheterocycle of formula (V)

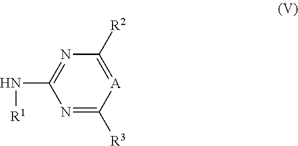

14. A compound of formula (II) and/or a salt thereof 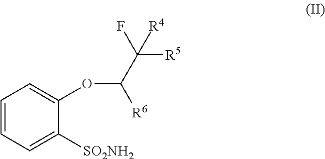
in which R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl.

15. A compound of formula (X) and/or a salt thereof 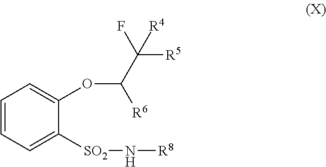
in which
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl;
and R⁸ is a branched C₁-C₈-group.

16. A compound of formula (VIII) and/or a salt thereof 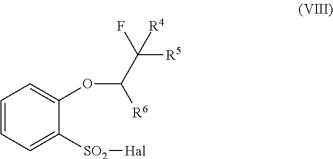
in which
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl;
and Hal is a halogen atom.

17. A compound of formula (IV) and/or a salt thereof 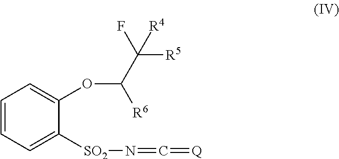
in which
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl
Q is selected from the group consisting of oxygen and sulfur.

18. A compound of formula (VI) and/or a salt thereof 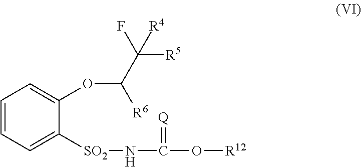
in which
R⁴ is selected from the group consisting of hydrogen, halogen and optionally substituted alkyl;
R⁵ is selected from the group consisting of hydrogen, halogen and optionally substituted alkyl;
R⁶ is selected from the group consisting of optionally substituted alkyl and optionally substituted alkenyl
R¹² is a substituted or unsubstituted (C₁-C₂₀)-hydrocarbon radical
Q is selected from the group consisting of oxygen and sulfur.

19. The compound and/or salt as claimed in any one of claim 14, wherein the compound and/or salt is present in a stereochemical purity of more than 50% to 100% in the (R) or (S) configuration with respect to the carbon atom marked with an (*):

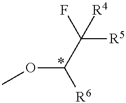

20. A composition comprising at least one compound of formula (I) and/or salt as claimed in claim 1.

21. The composition as claimed in claim 19, which comprises at least one further active compound selected from the group consisting of at least one further herbicide and at least one safener.

22. A herbicide and/or plant growth regulator comprising a compound of formula (I) and/or salt as claimed in claim 1 12 as herbicides and plant growth regulators.

23. A herbicide and/or plant growth regulator comprising a composition as claimed in claim 20.

24. A herbicide and/or plant growth regulator as claimed in claim 22 suitable for controlling plants in specific plant crops and/or as plant protection regulator.
